(19) United States
(12) Patent Application Publication
(10) Pub. No.: US 2026/0038638 A1
(43) Pub. Date: Feb. 5, 2026

(54) HIGH-THROUGHPUT PROTEOME MAPPING

(71) Applicant: The General Hospital Corporation, Boston, MA (US)

(72) Inventors: Wilhelm Haas, Charlestown, MA (US); Soroush Hajizadeh, Charlestown, MA (US)

(21) Appl. No.: 19/099,546

(22) PCT Filed: Jul. 28, 2023
(86) PCT No.: PCT/US2023/029015

§ 371 Date: Jan. 29, 2025

Related U.S. Application Data
(60) Provisional application No. 63/393,399, filed on Jul. 29, 2022.

(51) Int. Cl. G16B 40/10; G01N 33/68; G16B 30/00; G16B 40/20

(52) U.S. Cl. CPC G16B 40/10; G01N 33/6848; G16B 30/00; G16B 40/20

(57) ABSTRACT

Methods, systems, and apparatus, including computer programs encoded on computer storage media, for proteome mapping. One of the methods includes: identifying one or more target peptide sequences for a sample; estimating an elution order of one or more expected peptides from a chromatography column; and initiating generation of a first set of mass spectrometry spectra for the sample. The method also includes detecting peaks within the first set of mass spectrometry spectra to determine a real-time status with respect to the estimated elution order; selecting one or more peptide ions that are (i) observed in the first set of mass spectrometry spectra and (ii) included among the one or more peptides expected to be present in the sample; and initiating generation of a second set of mass spectrometry spectra for the one or more selected peptide ions.

### CROSS-REFERENCE TO RELATED APPLICATIONS

This application claims the benefit under 35 U.S.C. § 119 (e) of the filing date of U.S. Patent Application No. 63/393,399, for A Method of Targeted Plasma Proteomics, which was filed on Jul. 29, 2022, and which is incorporated here by reference.

### BACKGROUND

### Technical Field

This specification relates to mapping proteomes, e.g., proteomes of plasma or tissue.

### Background

The large-scale study of proteins in a proteome, sometimes referred to as “proteomics,” has many applications including the detection of various diagnostic markers, candidates for vaccine production, understanding pathogenicity mechanisms, alteration of expression patterns in response to different signals, and interpretation of functional protein pathways in different diseases. For example, proteomics can be used to identify and screen for biomarkers for diseases such as cancer, allowing for early detection.

The term “proteome” refers to the entire set of proteins and/or peptides that are, or can be, expressed by a genome, cell, tissue, or organism at a certain time. For example, a proteome of plasma can refer to the entire set of proteins that is, or can be, expressed in plasma. Similarly, a proteome of a particular human tissue can refer to the entire set of proteins that is, or can be, expressed in that particular human tissue.

Developing an understanding of the proteins and/or peptides within a particular proteome is important to the advancement of proteomics. This can be achieved, at least in part, through “proteome mapping,” which refers to the detection and identification of peptide and/or proteins within a proteome (e.g., by analyzing one or more samples of a relevant cell, tissue, plasma, etc.). In some cases, proteome mapping can also include the quantification of peptides and/or proteins within a sample. However, given the very large number of proteins in certain proteomes (e.g., thousands to millions of proteins), faster, more accurate, and more sensitive techniques for high-throughput proteome mapping are desired.

### SUMMARY

This specification describes technologies for high-throughput proteome mapping using liquid chromatography (LC) followed by multiple rounds of mass spectrometry (MS). For example, a first MS step, referred to herein as “MS1,” can be implemented to measure masses of intact peptides that are eluted into a mass spectrometer from a microcapillary chromatography column used in the LC process. A second MS step, referred to herein as “MS2” can generate measurements (e.g., spectra) by isolating one or more peptide ions as above, fragmenting the ions, and then identifying peptide sequences based on the resulting fragment ions, allowing identification of the original peptide ions. In some cases, a third MS step, referred to herein as “MS3” can be implemented to quantify peptides in the sample by generating measurements (e.g., spectra) indicative of isobaric mass tag reporter ions (e.g., tandem mass tag [TMT] reporter ions, isobaric tags for relative and absolute quantitation (iTRAQ), etc.) that correspond to isolated MS2 fragments at high sensitivity and accuracy.

For broad proteome coverage, existing approaches to proteome mapping using LC and MS typically involve fractionating a sample to create multiple fractions on which to perform MS1, and then selecting one or more peptide ions for a single-section MS2 spectra acquisition based on intensity measurements of the MS1 spectra. Such approaches are referred to sometimes as data-dependent acquisition [DDA] approaches.

Among other improvements to DDA techniques, the techniques described herein use (i) an intelligent sectioning approach to acquiring MS1 spectra based on predictions of which peptides are likely to be eluted at a particular retention time, (ii) an intelligent selection of peptide ions for MS2 spectra acquisition based on predictions of which peptides are likely to be eluted at a particular retention time, and (iii) intelligent windowing of MS2 spectra based on the results of previous MS1 scans in order to overcome the need for fractionating samples (thereby increasing the proteome mapping throughput for a given MS setup) and to increase sensitivity for peptide detection. The techniques disclosed herein also include improved approaches for identifying peptides from MS2 spectra (including identifying multiple peptides from a single MS2 spectra), and improved approaches for generating MS3 spectra (including techniques for selecting which peptide fragment ions to subject to MS3 scanning and techniques for optimizing ion accumulation time for MS3 spectra to achieve improved signal-to-noise ratio for peptide quantification).

In one aspect, a method is featured. The method includes identifying one or more target peptide sequences for a sample, the one or more target peptide sequences corresponding to one or more peptides expected to be present in the sample. The method also includes estimating, using one or more machine learning models, an elution order of the one or more expected peptides from a chromatography column; and initiating generation of a first set of mass spectrometry spectra for the sample. The method also includes, during generation of the first set of mass spectrometry spectra, detecting peaks within the first set of mass spectrometry spectra to determine a real-time status with respect to the estimated elution order. The method also includes initiating generation of a second set of mass spectrometry spectra for the one or more selected peptide ions.

Implementations can include the examples described below and herein elsewhere. In some implementations, identifying the one or more target peptide sequences for the sample can include predicting, using one or more additional machine learning models, fragment intensities of mass spectrometry spectra of a plurality of peptides; ranking the plurality of peptides based on a metric indicative of a variance of the predicted fragment intensities for each of the plurality peptides; and selecting a subset of the plurality of peptides that has the lowest values of the metric. In some implementations, the method can include estimating a compensation voltage that maximizes sensitivity of a mass spectrometer to the peptide ions. Selecting the one or more peptide ions that are (i) observed in the first set of mass spectrometry spectra and (ii) included among the one or more peptides expected to be present in the sample can be additionally based on the compensation voltage. In some implementations, the sample can be an unfractionated sample. In some implementations, the sample can be chemically tagged with an isobaric mass tag. In some implementations, initiating generation of the first set of mass spectrometry spectra for the sample can include generating a plurality of individual spectra having different mass-to-charge ranges. In some implementations, the different mass-to-charge ranges can be selected based on at least one of (i) the one or more target peptide sequences, (ii) the determined real-time status with respect to the estimated elution order, (iii) intensities of previously recorded signals in the given mass-to-charge ranges, or (iv) compensation voltage predictions. In some implementations, initiating generation of the second set of mass spectrometry spectra for the one or more selected peptide ions can include defining a width of a mass-to-charge range for at least one spectrum of the second set of mass spectrometry spectra, the width being defined based on (i) intensities of signals in the first set of mass spectrometry spectra, (ii) a number of peptide ion signals in a given mass-to-charge range, and (iii) an estimated accumulation time required for collecting a threshold number of ions for each of the peptide ion signals in the given mass-to-charge range. In some implementations, the method can include analyzing the second set of mass spectrometry spectra, wherein the analyzing includes inputting data indicative of the second set of mass spectrometry spectra into one or more convolutional neural networks trained to identify a presence of one or more peptides in the sample based on the data indicative of the second set of mass spectrometry spectra. In some implementations, the method can include selecting one or more fragment ions that are observed in the second set of mass spectrometry spectra; and initiating generation of a third set of mass spectrometry spectra for the one or more selected fragment ions. In some implementations, the third set of mass spectrometry spectra can be generated by (i) isolating the one or more selected fragment ions, (ii) further fragmenting the one or more selected fragment ions to produce further fragmented ions, and (iii) detecting at least a portion of the further fragmented ions, wherein the further fragmented ions comprise isobaric tag reporter ions. In some implementations, selecting the one or more fragment ions that are observed in the second set of mass spectrometry spectra can include scoring the one or more fragment ions based on at least one of: (i) a correlation between predicted and observed fragment ion intensities, (ii) a deviation between predicted and observed retention times for the one or more expected peptides, (iii) a number of observed fragment ions relative to a number of fragment ions predicted to be observed, (iv) a mass accuracy of an observed peptide signal from the first set of mass spectrometry spectra, and (v) a score reflecting a match between observed and predicted data based on a background-normalized dot-product. In some implementations, initiating the generation of the third set of mass spectrometry spectra for the one or more selected fragment ions can include: estimating a time required for collecting a threshold amount of each of the one or more selected fragment ions that correspond to a single peptide, the threshold amount corresponding to a signal-to-noise threshold for isobaric tag reporter ion signals; and initiating the generation of the third set of mass spectrometry spectra to collect data for at least the estimated time. In some implementations, the method can include analyzing the third set of mass spectrometry spectra for the one or more selected fragment ions to quantify an amount of at least one detected peptide present in the sample. In some implementations, the method can include monitoring a mass-to-charge ratio of intact peptide ions in the first set of mass spectrometry spectra. In some implementations, initiating generation of the second set of mass spectrometry spectra for the one or more selected peptide ions can include isolating the one or more selected peptide ions in a mass spectrometer that produces the mass spectrometry spectra, fragmenting the one or more selected peptide ions to generate fragment ions, and recording measurements related to at least a portion of the generated fragment ions.

In another aspect a system is featured. The system includes one or more computers and one or more storage devices on which are stored instructions that are operable, when executed by the one or more computers, to cause the one or more computers to perform operations. The operations include identifying one or more target peptide sequences for a sample, the one or more target peptide sequences corresponding to one or more peptides expected to be present in the sample; and estimating, using one or more machine learning models, an elution order of the one or more expected peptides from a chromatography column. The operations also include initiating generation of a first set of mass spectrometry spectra for the sample; and during generation of the first set of mass spectrometry spectra, detecting peaks within the first set of mass spectrometry spectra to determine a real-time status with respect to the estimated elution order. The operations also include, based on the determined real-time status with respect to the estimated elution order, selecting one or more peptide ions that are (i) observed in the first set of mass spectrometry spectra and (ii) included among the one or more peptides expected to be present in the sample; and initiating generation of a second set of mass spectrometry spectra for the one or more selected peptide ions.

Implementations can include the examples described below and herein elsewhere. In some implementations, identifying the one or more target peptide sequences for the sample can include predicting, using one or more additional machine learning models, fragment intensities of mass spectrometry spectra of a plurality of peptides; ranking the plurality of peptides based on a metric indicative of a variance of the predicted fragment intensities for each of the plurality peptides; and selecting a subset of the plurality of peptides that has the lowest values of the metric. In some implementations, the operations can include estimating a compensation voltage that maximizes sensitivity of a mass spectrometer to the peptide ions. Selecting the one or more peptide ions that are (i) observed in the first set of mass spectrometry spectra and (ii) included among the one or more peptides expected to be present in the sample can be additionally based on the compensation voltage. In some implementations, the sample can be an unfractionated sample. In some implementations, the sample can be chemically tagged with an isobaric mass tag. In some implementations, initiating generation of the first set of mass spectrometry spectra for the sample can include generating a plurality of individual spectra having different mass-to-charge ranges. In some implementations, the different mass-to-charge ranges can be selected based on at least one of (i) the one or more target peptide sequences, (ii) the determined real-time status with respect to the estimated elution order, (iii) intensities of previously recorded signals in the given mass-to-charge ranges, or (iv) compensation voltage predictions. In some implementations, initiating generation of the second set of mass spectrometry spectra for the one or more selected peptide ions can include defining a width of a mass-to-charge range for at least one spectrum of the second set of mass spectrometry spectra, the width being defined based on (i) intensities of signals in the first set of mass spectrometry spectra, (ii) a number of peptide ion signals in a given mass-to-charge range, and (iii) an estimated accumulation time required for collecting a threshold number of ions for each of the peptide ion signals in the given mass-to-charge range. In some implementations, the operations can include analyzing the second set of mass spectrometry spectra, wherein the analyzing includes inputting data indicative of the second set of mass spectrometry spectra into one or more convolutional neural networks trained to identify a presence of one or more peptides in the sample based on the data indicative of the second set of mass spectrometry spectra. In some implementations, the operations can include selecting one or more fragment ions that are observed in the second set of mass spectrometry spectra; and initiating generation of a third set of mass spectrometry spectra for the one or more selected fragment ions. In some implementations, the third set of mass spectrometry spectra can be generated by (i) isolating the one or more selected fragment ions, (ii) further fragmenting the one or more selected fragment ions to produce further fragmented ions, and (iii) detecting at least a portion of the further fragmented ions, wherein the further fragmented ions comprise isobaric tag reporter ions. In some implementations, selecting the one or more fragment ions that are observed in the second set of mass spectrometry spectra can include scoring the one or more fragment ions based on at least one of: (i) a correlation between predicted and observed fragment ion intensities, (ii) a deviation between predicted and observed retention times for the one or more expected peptides, (iii) a number of observed fragment ions relative to a number of fragment ions predicted to be observed, (iv) a mass accuracy of an observed peptide signal from the first set of mass spectrometry spectra, and (v) a score reflecting a match between observed and predicted data based on a background-normalized dot-product. In some implementations, initiating the generation of the third set of mass spectrometry spectra for the one or more selected fragment ions can include: estimating a time required for collecting a threshold amount of each of the one or more selected fragment ions that correspond to a single peptide, the threshold amount corresponding to a signal-to-noise threshold for isobaric tag reporter ion signals; and initiating the generation of the third set of mass spectrometry spectra to collect data for at least the estimated time. In some implementations, the operations can include analyzing the third set of mass spectrometry spectra for the one or more selected fragment ions to quantify an amount of at least one detected peptide present in the sample. In some implementations, the operations can include monitoring a mass-to-charge ratio of intact peptide ions in the first set of mass spectrometry spectra. In some implementations, initiating generation of the second set of mass spectrometry spectra for the one or more selected peptide ions can include isolating the one or more selected peptide ions in a mass spectrometer that produces the mass spectrometry spectra, fragmenting the one or more selected peptide ions to generate fragment ions, and recording measurements related to at least a portion of the generated fragment ions. In some implementations, at least one of the one or more computers can be included in a mass spectrometer.

In another aspect, one or more machine-readable storage devices are featured. The one or more machine-readable storage devices have encoded thereon computer readable instructions for causing one or more processing devices to perform operations. The operations include identifying one or more target peptide sequences for a sample, the one or more target peptide sequences corresponding to one or more peptides expected to be present in the sample; and estimating, using one or more machine learning models, an elution order of the one or more expected peptides from a chromatography column. The operations also include initiating generation of a first set of mass spectrometry spectra for the sample; and during generation of the first set of mass spectrometry spectra, detecting peaks within the first set of mass spectrometry spectra to determine a real-time status with respect to the estimated elution order. The operations also include, based on the determined real-time status with respect to the estimated elution order, selecting one or more peptide ions that are (i) observed in the first set of mass spectrometry spectra and (ii) included among the one or more peptides expected to be present in the sample; and initiating generation of a second set of mass spectrometry spectra for the one or more selected peptide ions.

Implementations can include the examples described below and herein elsewhere. In some implementations, identifying the one or more target peptide sequences for the sample can include predicting, using one or more additional machine learning models, fragment intensities of mass spectrometry spectra of a plurality of peptides; ranking the plurality of peptides based on a metric indicative of a variance of the predicted fragment intensities for each of the plurality peptides; and selecting a subset of the plurality of peptides that has the lowest values of the metric. In some implementations, the operations can include estimating a compensation voltage that maximizes sensitivity of a mass spectrometer to the peptide ions. Selecting the one or more peptide ions that are (i) observed in the first set of mass spectrometry spectra and (ii) included among the one or more peptides expected to be present in the sample can be additionally based on the compensation voltage. In some implementations, the sample can be an unfractionated sample. In some implementations, the sample can be chemically tagged with an isobaric mass tag. In some implementations, initiating generation of the first set of mass spectrometry spectra for the sample can include generating a plurality of individual spectra having different mass-to-charge ranges. In some implementations, the different mass-to-charge ranges can be selected based on at least one of (i) the one or more target peptide sequences, (ii) the determined real-time status with respect to the estimated elution order, (iii) intensities of previously recorded signals in the given mass-to-charge ranges, or (iv) compensation voltage predictions. In some implementations, initiating generation of the second set of mass spectrometry spectra for the one or more selected peptide ions can include defining a width of a mass-to-charge range for at least one spectrum of the second set of mass spectrometry spectra, the width being defined based on (i) intensities of signals in the first set of mass spectrometry spectra, (ii) a number of peptide ion signals in a given mass-to-charge range, and (iii) an estimated accumulation time required for collecting a threshold number of ions for each of the peptide ion signals in the given mass-to-charge range. In some implementations, the operations can include analyzing the second set of mass spectrometry spectra, wherein the analyzing includes inputting data indicative of the second set of mass spectrometry spectra into one or more convolutional neural networks trained to identify a presence of one or more peptides in the sample based on the data indicative of the second set of mass spectrometry spectra. In some implementations, the operations can include selecting one or more fragment ions that are observed in the second set of mass spectrometry spectra; and initiating generation of a third set of mass spectrometry spectra for the one or more selected fragment ions. In some implementations, the third set of mass spectrometry spectra can be generated by (i) isolating the one or more selected fragment ions, (ii) further fragmenting the one or more selected fragment ions to produce further fragmented ions, and (iii) detecting at least a portion of the further fragmented ions, wherein the further fragmented ions comprise isobaric tag reporter ions. In some implementations, selecting the one or more fragment ions that are observed in the second set of mass spectrometry spectra can include scoring the one or more fragment ions based on at least one of: (i) a correlation between predicted and observed fragment ion intensities, (ii) a deviation between predicted and observed retention times for the one or more expected peptides, (iii) a number of observed fragment ions relative to a number of fragment ions predicted to be observed, (iv) a mass accuracy of an observed peptide signal from the first set of mass spectrometry spectra, and (v) a score reflecting a match between observed and predicted data based on a background-normalized dot-product. In some implementations, initiating the generation of the third set of mass spectrometry spectra for the one or more selected fragment ions can include: estimating a time required for collecting a threshold amount of each of the one or more selected fragment ions that correspond to a single peptide, the threshold amount corresponding to a signal-to-noise threshold for isobaric tag reporter ion signals; and initiating the generation of the third set of mass spectrometry spectra to collect data for at least the estimated time. In some implementations, the operations can include analyzing the third set of mass spectrometry spectra for the one or more selected fragment ions to quantify an amount of at least one detected peptide present in the sample. In some implementations, the operations can include monitoring a mass-to-charge ratio of intact peptide ions in the first set of mass spectrometry spectra. In some implementations, initiating generation of the second set of mass spectrometry spectra for the one or more selected peptide ions can include isolating the one or more selected peptide ions in a mass spectrometer that produces the mass spectrometry spectra, fragmenting the one or more selected peptide ions to generate fragment ions, and recording measurements related to at least a portion of the generated fragment ions.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1 is a diagram illustrating proteome mapping-related processes, applications, and results.

FIG. 2 is a diagram illustrating a process for proteome mapping.

FIG. 3 is a plot showing a relationship between number of peptide identifications by mass spectrometry and intra-protein-observability (IPO) score.

FIG. 4A is a plot showing distributions of the number of peptide MS1 signals detected by (i) single-section MS1 spectra and (ii) 10-section MS1 spectra.

FIG. 4B is an example spectrum from a single-section MS1 scan.

FIG. 4C is an example spectrum from a 10-section MS1 scan.

FIG. 5 is a plot showing a comparison between observed and predicted signal intensities for a peptide ion.

FIG. 6 is a plot showing a relationship between observed peptide retention time and predicted peptide elution order.

FIG. 7 is a plot showing a distribution of the deviation between predicted and measured ion mobility compensation voltages (CV).

FIG. 8 is a plot showing distributions of combined MS1 targeted peptide signal scores for successful versus unsuccessful MS2 assignments.

FIG. 9A is a plot showing a number of successful peptide assignments from various 10 m/z isolation width MS2 spectra generated at two resolution levels.

FIG. 9B is a plot showing maximum m/z distances between correctly assigned MS1 peptide signals versus maximum m/z distances between MS1 peptide signals for various 10 m/z isolation width MS2 spectra.

FIG. 10 is a plot showing observed versus predicted normalized peptide fragment ion intensities for various peptide ions.

FIGS. 11A-11B are plots showing distributions of validation score values for true positive MS2 assignments and false positive MS2 assignments.

FIG. 12A is a diagram of a neural network-based model.

FIG. 12B is a plot comparing results from assigning peptides to MS2 spectra using an XCorr-scoring approach and a neural network-based approach.

FIG. 13 is a plot comparing signal-to-noise ratios achieved for peptide quantification using MS3 spectra obtained through various approaches.

FIG. 14 is a flowchart of a process for mapping a proteome.

FIG. 15 is a diagram illustrating an example of a computing environment.

Like reference numbers and designations in the various drawings indicate like elements.

### DETAILED DESCRIPTION

Disclosed herein is a mass spectrometry-based method for high-throughput mapping of proteomes (e.g., plasma proteomes, cell proteomes, tissue proteomes, etc.). One of the intended uses of the method is the identification and screening for biomarkers including markers for early detection of cancer and other diseases. Among other novel features, one feature is a real-time highly accurate prediction of peptide retention times that allows for targeted mass spectrometry-based proteomics of plasma proteome (or any other proteome samples) at a throughput more than 10 times better than currently available while reaching a plasma proteome coverage that is at least 2 times two times better than that provided by state-of-the-art methods. This will allow for early disease detection. Cancer detection is an example of a promising application, but this method will be broadly applicable to detection of other diseases and/or other applications where information about proteomes are useful.

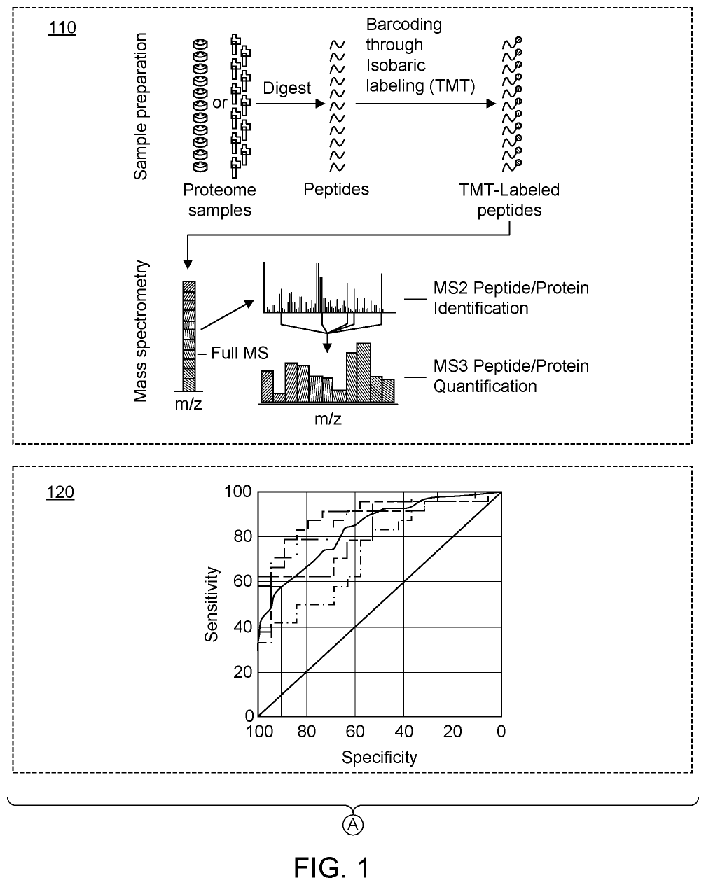

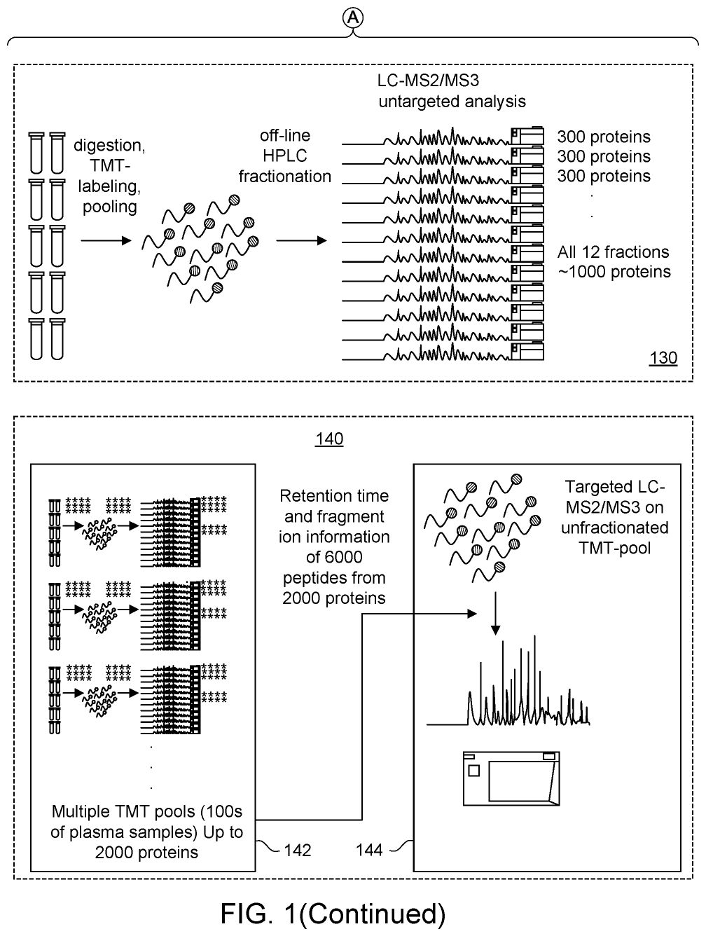

Referring to FIG. 1, box 110 shows an example of multiplexed mass spectrometry-based proteomics using TMT reagents and LC-MS2/MS3 (e.g., a proteome mapping process that includes liquid chromatography [LC] followed by multiple mass spectrometry steps [MS1, MS2, and MS3]). Box 110 shows eleven samples being quantified simultaneously.

In box 120, a plot is shown demonstrating that a set of ten plasma protein biomarkers identified by quantitative proteomics can distinguish lung cancer cases and high-risk controls with a sensitivity of 58% at a specificity threshold of 90%. This represents an example of how the results of proteome mapping (e.g., identified protein biomarkers) can be applied to disease detection.

In box 130, an LC-MS2/MS3 approach to plasma proteome mapping based on an untargeted selection of peptide ions is shown. As described above, this approach includes off-line fractionation of pooled plasma samples into twelve fractions to reach a depth of about 1000 quantified proteins.

In box 140, sub-box 142 shows how mapping hundreds of plasma proteomes (e.g., using the approach shown in box 130) can yield a training dataset of LC, MS1, MS2, and MS3 results, including a list of more than 2000 identified plasma proteins. The fragment ion and retention time information of peptides quantified in these runs are included in the training dataset and can be used to build a targeted LC-MS2/MS3 plasma proteome mapping method that can allow for the quantification of all 2000 proteins from a single LC-MS2/MS3 run (e.g., a LC-MS2/MS3 run on a single unfractionated sample), improving the sample throughput by a factor of more than 10.

As described in further detail herein, a proteome mapping technique can use multiplexed quantitative proteomics (e.g., as described in PMID: 12713048, PMID: 21963607, PMID: 24927332, PMID: 32203386). As shown in boxes 110, 130, and 140 of FIG. 1, multiplexing can be achieved using tandem mass tag (TMT) reagents as in the above-referenced papers or other reagents such as iTRAQ (see, e.g., PMID: 15385600). Multiplexing in proteomics is analogous to barcoding. It allows the simultaneous quantification of multiple samples—currently up to 18 (see, e.g., PMID: 33900084)—in one analysis. In the proteome mapping techniques described herein (and as shown in box 110 of FIG. 1), a method for accurate multiplexed quantification is implemented, including the use of MS3 mass spectrometry scans. Existing MS3 techniques are described, for example, in

PMID: 21963607 and PMID: 24927332. As shown in box 110, the method includes a full-MS experiment to measure the masses of intact peptides (referred to as MS1), followed by additional mass spectrometry experiments to sequence-specific peptide ions (referred to as MS2), followed by further mass spectrometry experiments to accurately quantify the peptides (referred to as MS3).

As shown in box 130 of FIG. 1, conventional techniques for proteome mapping of plasma samples (used herein as a representative example of a type of sample) include pooling the barcoded tryptic digests of the plasma samples, and then fractionating this pool to allow mapping the proteome at greater depth. For example, in an experimental setup that allows for analyzing 12 fractions per sample pool, the entire sample set can be analyzed in 36 hours. Using this setup and simultaneously analyzing 16 samples (e.g., using TMT-labeling), the mass spectrometry time per sample is about 2.25 hours. Each fraction is subjected to nano-capillary chromatography coupled to the mass spectrometer. Each peptide enters the mass spectrometer at different times (referred to as “retention times”) based on the peptide sequence and the chromatographic system. Using such conventional techniques, the depth of analysis is about 1000 proteins. However, the plasma proteome is believed to contain more than 3000 proteins (see, e.g., PMID: 28938075). The enormous range of protein concentrations in plasma therefore poses a substantial challenge to mapping the entire plasma proteome using the approach shown in box 130 of FIG. 1.

The mass spectrometry approach illustrated in box 130 is based on real-time data-dependent detection of peptide ions that are then subjected to automated further analyses in the mass spectrometer. The real-time detection leads to partly random sampling, and the peptides and proteins quantified when analyzing the same pool of samples (e.g., 12 fractions of sample) twice will be different. When analyzing, e.g., 10 TMT sets of 16 samples each, the total number of quantified proteins could exceed 2000.

Compared to the mass spectrometry approach illustrated in box 130, this specification discloses improved approaches to proteome mapping that could enable quantification of all 2000 proteins in each TMT set not by analyzing 12 sample fractions but by analyzing only a single unfractionated sample (e.g., taking less than 10 minutes per sample if 18-plexing). An unfractionated sample analysis approach is shown in sub-box 144 of box 140 (shown in FIG. 1). Unfractionated sample analysis approaches, as described herein, can be achieved using a targeted proteomics method where one pre-defines which peptides will be quantified. Targeting peptides for quantification is typically extremely difficult if the intact peptide signal is below the noise level, as will be the case for the many of the 2000 and more proteins in the plasma sample that one might want to quantify. One lab has published an elegant way to overcome this hurdle using multiplexed proteomics (see, e.g., PMID: 28065596). Their method uses synthetic peptides to generate guide-signals that inform the mass spectrometer of the exact retention time of a peptide and, thereby, cause the mass spectrometer to amplify the below-noise-level signal to quantify the peptide. Unfortunately though, the number of target peptides is limited using this approach. The current highest number of targeted peptides is 520 peptides from 260 proteins (see, e.g., PMID: 32332170). Quantifying 2000 proteins using a synthetic peptide approach would require the use of at least 4000 peptides to achieve the quantification of multiple peptides per protein.

The proteome mapping techniques described in this specification overcome the need to use synthetic peptides for generating guide-signals. Rather than using synthetic peptides to generate guide-signals, the techniques described herein involve predicting, in real-time, the exact retention time of peptides based on the retention time of pre-eluding peptides (e.g., precursor peptides that have already been eluded from a chromatography column into the mass spectrometer). Using large training datasets derived from previous liquid chromatography and mass spectrometry experiments (e.g., experiments performed on hundreds of plasma samples, as shown in sub-box 142 of box 140 shown in FIG. 1), real-time prediction of peptide elution order (and subsequently, peptide retention times) can be made. Confidently assigned peptides (e.g., by MS2) will be used as standards to accurately predict the elution order and/or retention times of upcoming peptides. By using real-time predictions and predicted elution order, the techniques described herein have the advantage of being robust, with degradation of the chromatographic column or other column changes having little to no effect on the prediction quality as long as the same chromatographic material is used.

As described in further detail herein, the MS2 fragment ions best suited for accurate quantification using the MS3 experiment can be preselected using a machine learning-based approach. In this machine learning-based approach, one or more machine learning models (e.g., neural networks) can be used, with the one or more machine learning models being trained on a training dataset derived from previously performed liquid chromatography and/or mass spectrometry experiments.

In one implementation of the proteome mapping methods described herein, the proteome mapping starts with analyzing high-intensity peptide ions using a data-dependent method (e.g., similar to the data-dependent methods previously described in relation to box 130 of FIG. 1). However, once peptides are confidently identified, their signals in the full-MS spectra (e.g., MS1 spectra) are traced to identify the apex of the chromatographic peaks. This apex information is used to predict the apexes of subsequently eluded targeted peptide ions. At the predicted apex of each targeted peptide, an MS2 spectrum is performed on a number of ions that allow for the detection of the peptide, even if the intact peptide signal is under the noise level for the full-MS1 spectra. The MS2 spectrum is monitored for the predicted peptide-specific MS2 fragment ions, and a statistical method is used to calculate the likelihood of the peptide ion being present. If the peptide ion is found to be present, an MS3 spectrum on the preselected fragment ions is then performed. Peptide ions with high full-MS intensity (e.g., MS1 intensity) are used across the entire chromatogram for real-time retention time prediction to address eventual retention time shifts during the chromatographic separation. In some cases, the implementation of proteome mapping just described can be combined with ion mobility mass spectrometry, as further described herein. Ion mobility mass spectrometry is gaining in importance for analyzing complex proteome samples (see, e.g., PMID: 30672687), and it is a promising tool to increase the analytical depth of mass spectrometry-based plasma proteomics.

Early cancer detection saves lives, and there is a high demand for cost-effective blood-based screening methods to enable early diagnosis of cancer. Currently, large efforts are underway to detect cancer from blood samples (e.g., liquid biopsies) by monitoring for cancer-specific mutations in circulating tumor DNA (ctDNA). Although the results of these efforts are very promising, it is yet not clear if ctDNA can enable detection of pre-symptomatic patients and very small localized tumors. Furthermore, the identification of ctDNA driver mutations does not indicate the identity of the tumor, which complicates early intervention, and ctDNA analysis does not enable one to distinguish between benign and malignant lesions carrying the driver mutations, which carries the risk of overtreatment upon ctDNA mapping.

Mapping blood plasma proteomes to identify cancer biomarkers has the potential to overcome problems with ctDNA analysis alone. A holistic map of plasma proteins may not only enable (i) detection of cancer by identifying tumor-specific markers, but also (ii) locating the tumor through identifying tumor-leaking tissue-specific proteins, and (iii) distinguishing between benign and malignant lesions (e.g., through overall changes in the plasma proteome indicating inflammations or other systemic dysregulations). In addition, plasma proteome changes may also be used as biomarkers for other diseases besides cancer.

Mass spectrometry (MS) is a powerful analytical tool for unbiased mapping of plasma proteomes, and MS's potential for identifying disease biomarkers from plasma has driven numerous research efforts for early cancer detection. However, the success of these efforts has been limited, partially due to historical technological shortcomings of MS approaches, which were overcome only recently. Historically, the use of mass spectrometry for biomarker identification was limited by sensitivity thresholds of MS technologies and by the high cost of proteome mappings. Thus, instead of mapping whole proteomes of plasma samples, markers have previously been identified by determining proteome differences between small numbers of tumors and healthy tissue samples, and the data from these studies were often unsuccessfully extrapolated to predict potential protein markers that were leaked into the bloodstream.

Today, the recent development of high-throughput mass spectrometry through multiplexing (e.g., shown in box 110 of FIG. 1) allows for directly mapping plasma proteomes to a depth of up to 1000 proteins for biomarker detection. The same strategy of unbiased proteome mapping can be used for biomarker discovery and validation and has the potential to also be used for cancer screening. The analysis only requires about 5 μl of plasma, and, therefore, facilitates the curation of test samples for developing assays from plasma banks.

As an illustrative example, this high-throughput mass spectrometry with multiplexing has been used to develop an assay to support the use of low dose CT (LDCT) scans for early lung cancer detection. Plasma samples were collected from hospital patients with negative screening LDCT scans (high-risk controls, all with >30 pack-years of smoking) as well as pre-operative samples from patients undergoing resection of early-stage lung cancer (cases, also with positive smoking history, >60% stage I tumors). Multiplexed quantitative proteomics was then used to map the plasma proteome of 48 early-stage lung cancer cases and 38 high-risk controls. By splitting the data randomly into training and validation sets five times, a 10-protein biomarker panel was identified with a median area under the curve (AUC) of 0.83 (95% confidence interval: 0.70-0.95) (box 120 of FIG. 1).

These promising data have evoked interest in using high-throughput mass spectrometry for other early cancer detection projects such as identifying a plasma biomarker set to distinguish between low and high-grade intraductal papillary mucinous neoplasm (IPMN). In the IPMN context, the goal is to develop a blood-based assay that allows directing the timing of surgical intervention before the formation of invasive pancreatic ductal adenocarcinoma (PDAC) from high-grade IPMN. Furthermore, experiments have been initialized to identify plasma-based biomarkers to support breast cancer screening through mammography by reducing the number of unnecessary breast biopsies without compromising early cancer detection.

A remaining hurdle to using high-throughput multiplexed mass spectrometry for plasma proteomics is the current limitation of sample throughput. It is estimated that the development of a very good biomarker set requires the mapping of about 1000 plasma samples per cancer type, and the current throughput limitations of high-throughput multiplexed mass spectrometry may be insufficient to keep up with the demand for plasma proteome mappings. The proteome mapping technologies disclosed herein improve upon existing high-throughput multiplexed mass spectrometry approaches, substantially increasing the throughput of plasma proteome mappings and decreasing the costs per sample analysis by at least two-fold. Such improvements can be catalytic in enabling many more early cancer detection projects at a fraction of the time and cost.

In an example conventional high-throughput multiplexed mass spectrometry approach, plasma proteins are digested with proteases and then each digest is labeled with a tandem mass tag (TMT) reagent (e.g., one out of up to eleven TMT reagents) that provide a barcoding functionality for quantifying the labeled samples simultaneously (shown in box 130 of FIG. 1). The labeled digests are then pooled and subjected to fractionation by regular high-performance liquid chromatography (HPLC) (or another off-line fractionation technique). The resulting fractions (e.g., twelve fractions in this example) are analyzed by mass spectrometry, which includes another fractionation by nano-capillary HPLC immediately before the peptides are injected into the mass spectrometer (LC-MS). Each fraction is analyzed for three hours resulting in a total analysis time of 36 hours per pooled sample set and, therefore, less than four hours of mass spectrometer time is used to map one plasma proteome. The analysis consists of repeating experimental cycles lasting for up to 5 seconds which are initiated by a full-MS screen (e.g., a MS1 screen) determining the intact masses of the peptide ions eluted off the nano-capillary column at the time of measurement. Peptides with the most intense signals in the full-MS spectra are then selected for MS2 scans that result in the identification of the amino acid sequences of the peptides, and a subset of the fragment ions identified in the MS2 scans are in turn selected for MS3 scans that reveal the concentration of the peptides across all analyzed samples. All these steps are performed in an automated manner, and a typical 3-hour MS run produces more than 20,000 pairs of MS2 and MS3 spectra of which only a fraction results in successful peptide identifications and quantifications. Peptides measurements are then combined to generate a list of quantified proteins. The number of quantified proteins per three-hour run is about 300 and about 1000 across all twelve runs (corresponding to the twelve sample fractions in this example) since there is an overlap of proteins quantified in each run.

In this conventional high-throughput MS approach, the off-line fractionation is implemented since the plasma proteome is dominated by a small number of highly abundant proteins, and without offline fractionation, peptides of these proteins would mask peptides of other proteins in the mixture. The signal intensities of peptides corresponding to less abundant proteins would be below the signal-to-noise level so that they are not selected for MS2 and MS3 scans and, therefore, would not be quantified.

It is also important to note that, in the conventional high-throughput MS approach shown in box 130, the automated selection process of peptide ions for MS2 and MS3 has a stochastic component, which is largest for peptide ions with an intensity close to the signal-to-noise level. Therefore, the peptides and proteins quantified in two analyses of the same sample will not be the same. As a consequence, when hundreds of samples are split up into TMT pools that each include multiple samples and are then analyzed, the number of proteins quantified for each TMT pool will be about 1,000. However, the number of proteins quantified in all of the TMT pools (e.g., overlapping quantification across all samples) will be lower, causing missing values since some proteins are not quantified in all of the samples. At the same time, the partially stochastic selection of peptide ions results in a number of proteins quantified across all TMT pools (not necessarily overlapping across all samples) that can be substantially higher than the number quantified for each pool. For example, in some experiments, more than 2,000 proteins were quantified across ten different TMT pools.

Compared to the conventional approach to high-throughput multiplexed MS shown in box 130, the improved proteome mapping technologies disclosed herein reduce the number of fractions that must be analyzed per TMT pool to increase sample throughput (as shown, for example, in sub-box 144 of FIG. 1). At the same time, the proteome mapping technologies disclosed herein increase the number of quantified proteins and the overlap of quantified proteins between TMT pools by reducing missing values. Instead of using a stochastic peptide-intensity based method to select peptide ions for MS2 and MS3 the improved proteome mapping technologies disclosed in this specification use a targeted peptide selection procedure. The target proteins are selected from proteins most commonly quantified in hundreds of plasma proteome datasets already acquired in the lab using conventional approaches (as shown in sub-box 142 of FIG. 1). The proteome maps can be derived from patient samples, and in the results presented herein, the patient samples have primarily come from lung cancer patients, and control plasma samples. However, it is understood and envisioned that other kinds of samples can be used. As more proteomes are mapped (including proteomes for other sample types), the list of target proteins can be readily updated or extended to include proteins more frequently found in plasma samples from patients of other cancer types.

### Artificial Intelligence-Powered MS for Ultrahigh Throughput Proteome Mapping

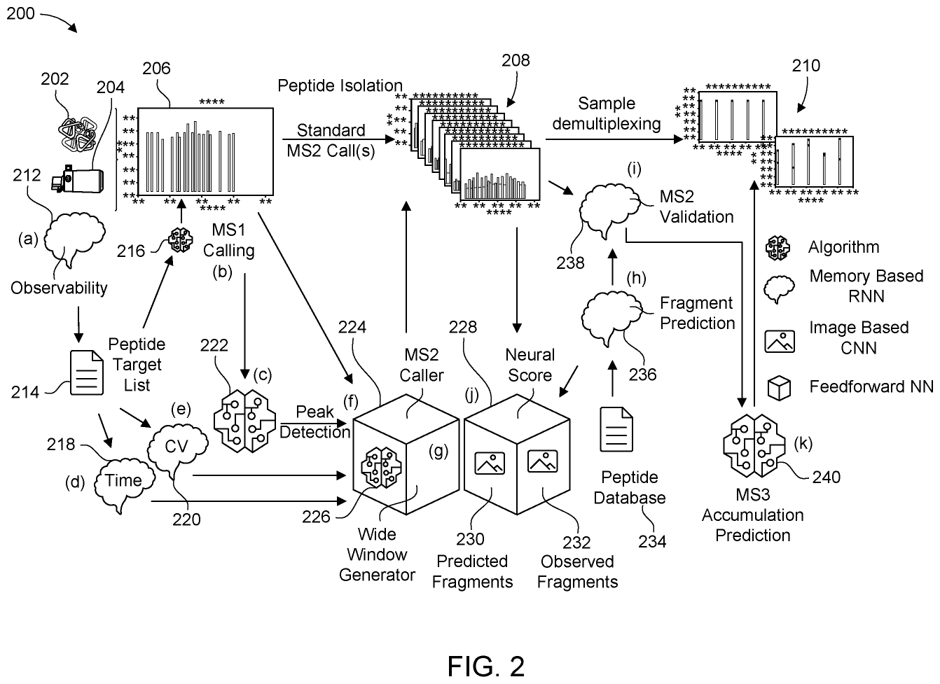

FIG. 2 illustrates an example process 200 for proteome mapping that yields various advantages (e.g., increased throughput, increased sensitivity, etc.) compared to existing high throughput multiplexed MS approaches. In particular, the process 200 includes the implementation of various neural networks—trained on millions of peptide mass spectra—to increase analysis throughput and analytical depth compared to existing approaches. The process 200 also provides for ultrahigh throughput proteome mapping by only monitoring peptides from a predefined list, making the process 200 a “targeted” proteomics approach. The process 200 also includes barcoding samples (e.g., using tandem-mass-tag (TMT) technology) to analyze multiple samples in parallel (e.g., up to 18 samples).

It is noted that while examples of neural network implementations are described throughout this specification, in some implementations, one or more other machine learning models may be applied. In general, whenever neural networks are described throughout this specification, it is envisioned that one or more other machine learning models can be implemented to accomplish a similar function. For example, these machine learning models can include models that employ decision trees, linear regression, multinomial logistic regression, Naive Bayes (NB), trained Gaussian NB, NB with dynamic time warping, multiple linear regression, Shannon entropy, support vector machine (SVM), one versus one support vector machine, k-means clustering, Q-learning, temporal difference (TD), neural networks, deep adversarial networks, and/or the like. In addition, unless otherwise specified, it is understood that the machine learning models can be trained using supervised learning approaches, semi-supervised learning approaches, reinforcement learning approaches, active learning approaches, continual learning approaches, and/or the like.

The process 200 starts with a mixture including one or more proteins 202 (e.g., one or more proteins present in a plasma or tissue sample) and a mass spectrometer 204 (e.g., a Thermo Fisher Scientific® Orbitrap Eclipse™ Tribrid™ mass spectrometer) to produce a first set of MS1 spectra including MS1 spectrum 206. The MS1 spectra measure masses of intact peptides that are eluted into the mass spectrometer 204 from a chromatography column (e.g., as the result of implementing a liquid chromatography process on a plasma or tissue sample). In some implementations, to enable multiplexing (as described above), the one or more proteins 202 can be tagged prior to the start of the process 200, e.g., using isobaric tags such as tandem-mass-tags (TMT), isobaric tags for relative and absolute quantification (iTRAQ), or any other isobaric tags. Importantly, however, offline fractionation of the mixture including one or more proteins 202 is not required prior to the start of the process 200.

After at least one MS1 spectrum (e.g., the MS1 spectrum 206) is generated, the process 200 includes generating MS2 spectra 208 (e.g., using the mass spectrometer 204) by isolating one or more peptide ions identified in the MS1 spectra and fragmenting the peptide ions to generate fragment ions that are used to identify the peptide sequences corresponding to the peptide ions. The process 200 further includes generating MS3 spectra 210 (e.g., using the mass spectrometer 204) by isolating fragment ions identified in the MS2 spectra 208 to generate further fragmented ions including TMT reporter ions at high sensitivity and accuracy. The MS3 spectra 210 can be acquired on single or multiple fragment ions for accurate isobaric tag-based quantification.

Unlike conventional approaches for high-throughput multiplexed MS, the process 200 does not automatically select peptide ions having the highest intensity measurements in the MS1 spectra for further MS2 scans. Instead, a number of machine learning (ML) and artificial intelligence (AI)-driven modules (e.g., software modules that implement one or more ML and/or AI algorithms) are implemented to direct the data acquisition of the MS1, MS2, and MS3 scans to optimize sample throughput and sensitivity.

As shown in FIG. 2, the process 200 includes (a) predicting which tryptic peptides from any protein sequence are the most likely ones to be identified in a MS-based proteomics experiment (e.g., implemented by observability module 212, which outputs peptide target list 214); (b) initiating generation of a MS1 spectrum (e.g., using MS1 calling algorithm 216); (c) detecting and matching targeted peptide sequences with intact peptide ion signals from the MS1 spectra (e.g., using the peak detection algorithm 222); (d) predicting (e.g., using time module 218) an elution order (and relatedly, a retention time) of peptides on the target peptide list 214; (e) predicting (e.g., using the CV model 220) the expected ion mobility compensation voltage (CV) in an ion mobility device (e.g., a Thermo Fisher Scientific® High-Field Asymmetric Waveform Ion Mobility Spectrometry device [“FAIMS”]) that maximizes sensitivity of the mass spectrometer 204 to the peptide ions; and (f) selecting which peptide ions to further analyze with MS2 scans (e.g., using the MS2 caller module 224). MS2 caller module 224 can include a wide window generator algorithm 226 that defines the isolation width for isolating the peptide ions for MS2 scans (allowing multiple peptides to be isolated simultaneously to accelerate the proteome mapping process). The process 200 also includes (h) predicting (e.g., using fragment prediction module 236 trained on peptide database 234) the intensity of fragment ion measurements in the MS2 spectra 208 of particular peptide ions at a specific collision energy; (i) generating (e.g., using MS2 validation module 238) a score to identify peptide fragment ion signals that are selected for MS3 scanning (including generating scores for multiple peptides from a singular MS2 spectra); and (j) identifying peptides present in the sample based on the generated MS2 spectra 208 (e.g., using the neural score module 228) by validating the matching of MS2 spectra 208 with peptide amino acid sequences. The neural score module 228 can be implemented using a predicted fragments module 230 (e.g., an image-based convolutional neural network [CNN]) and an observed fragments module 232 (e.g., another image-based CNN) that receive, as inputs, one or more MS2 spectra 208 and the predictions from the fragment prediction module 236. In some implementations, the neural score module 229 can allow for the identification of multiple peptides from a singular MS2 spectrum. The process 200 can also include predicting (e.g., using MS3 accumulation prediction algorithm 240) an ion accumulation time that should be used for generating the MS3 spectra 210 in order to achieve a desired signal-to-noise ratio for quantification. In some implementations, the MS3 accumulation prediction algorithm 240 can be implemented using a generalized random forest model, although in other implementations, one or more other machine learning models can be employed.

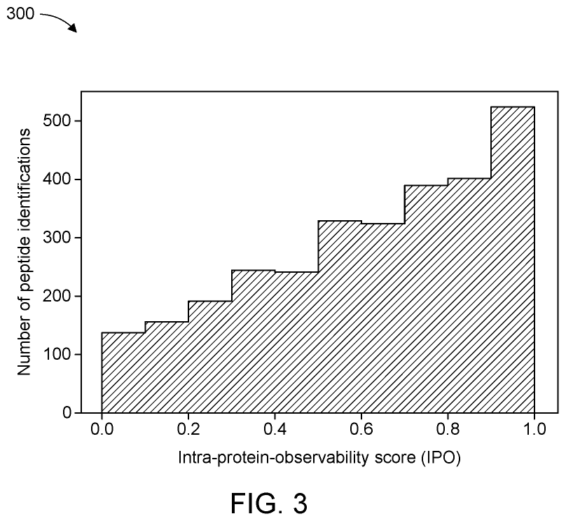

Having provided an overview of the process 200, further implementation details of the process 200 are described herein. In some implementations, the process 200 simply requires, as input, a list of peptides to be quantified (e.g., the peptide target list 214). The peptide target list 214 can be generated based on preliminary data acquired to define the proteome in the studied samples. However, in some cases, the peptide target list 214 can be generated as an output of the observability module 212. For example, the observability module 212 can be a neural network trained using data from previously conducted MS experiments such that the observability module is able to generate the peptide target list 214 simply based on a peptide database such as the peptide database 234. In particular, the observability module 212 can be built on top of a fragment prediction module (e.g., the fragment prediction module 236), so that the peptide target list 214 is generated based on one or more properties of the predicted fragment ion intensities for one or more peptides in the peptide database 234. In one implementation, the observability module 212 can calculate an “intra-protein-observability” (IPO) score based on the outputs of the fragment prediction module 236, wherein the IPO score is indicative of a variance of intensities across all predicted fragment ions. Typically, the smaller the variance (corresponding to a higher IPO score), the higher is the likelihood that the peptide is identified by mass spectrometry (as described in greater detail below in relation to FIG. 3). Thus, the observability module 212 can generate the peptide target list 214 by selecting the peptides from the peptide database 234 that have the highest IPO scores.

Once the peptide target list 214 is generated, for each of the peptides on the peptide target list 214, the elution order, CV (compensation voltage), and intensities can be predicted in advance of initiating any MS experiments to allow fast accessibility in real-time during the MS data acquisition. For example, the time module 218 (e.g., a neural network trained on previously acquired MS experimental data) can make an initial prediction of the elution order and/or corresponding retention times for the peptides on the peptide target list 214 (referred to herein as “target peptides”). The CV module 220 (e.g., another neural network trained on previously acquired MS experimental data) can similarly make an initial prediction of the CVs that will maximize the sensitivity of the mass spectrometer 204 to the target peptides, and enable identification of MS1 target peptide signal candidates based on their corresponding intensity distribution across various CV settings. Furthermore, a fragment prediction module (e.g., the fragment prediction module 236, which is implemented using another neural network trained on previously acquired MS experimental data) can make an initial prediction of the signal intensities of the acquired MS2 spectra likely to be obtained for the target peptides. In some cases, MS1 intensity signals for the target peptides can also be predicted at this stage (e.g., based on quantifying the heavy 13C isotopes present in the target peptide, as described in further detail below). After these initial predictions are made, all subsequent scoring and accumulation predictions in the process 200 are then performed in real-time as MS data acquisition occurs.

Once the first MS run begins (e.g., upon being called by the MS1 calling algorithm 216), the peak detection module 222 starts searching for MS1 peptide signals that correspond to the predicted chromatographic elution order/time (e.g., the output of time module 218), and the predicted CV (e.g., the output of the CV module 220). A list with then be filled with possible masses to scan for, based on the overlap of predicted masses and observed masses in the acquired MS1 spectra (e.g., MS1 spectrum 206). Multiple CVs are constantly stored in the MS1, and a predicted precursor distribution method is used to determine if a peak is the precursor peak for any of the targeted peptides which are ultimately called. In other words, if a distinctive (e.g., high-intensity) MS1 peak is expected just before the elution of a target peptide (e.g., a target peptide having a corresponding MS1 peak that would otherwise be below the noise level), once the distinctive MS1 peak is observed by the peak detection module 222, the MS2 caller module 224 (e.g., a feedforward neural network that receives elution time/order predictions, CV predictions, and peak detection information as inputs) can anticipate the subsequent elution of the target peptide and time an MS2 scan to acquire a MS2 spectra corresponding to the target peptide. Using this approach, the MS2 caller module 224 generates MS2 spectra for each peptide ion MS1 signal matching the peptide target list 214 cross-referenced with real-time predictions of elution time and CVs. Importantly, the time module 218 and/or the CV module 220 can be implemented to incorporate information from MS1 scans acquired in real-time to generate updated real-time predictions of elution order/time and CVs. For example, identified and quantified peptide signals can be used in real-time to adjust the retention time prediction and mass accuracy deviations of the mass spectrometer. This can prevent the deterioration of prediction quality as the process 200 progresses and has the additional advantage of making the predictions robust to degradation of the chromatographic column or other column changes that may occur.

In the process 200, as MS2 spectra 208 are being generated, MS2 information can be assigned to peptide amino acid sequences (sometimes in real-time). For example, the assignment of MS2 information can be achieved with the neural score module 228 (e.g., implemented as a feedforward neural network) that produces a neural score that allows comparison of the acquired MS2 information (e.g., from the MS2 spectra 208) with that of potential matching peptides' spectra predicted by the fragment prediction module 236 (which may also be a neural network). To produce the neural score, the neural score module 228 can, in some cases, include a first image-based CNN (e.g., predicted fragments module 230) that analyzes the predicted spectra output by the fragment prediction module 236, and a second image-based CNN (e.g., observed fragments module 232) that analyzes the acquired MS2 spectra 208. In other cases, the neural score can be calculated using a correlation-based comparison (e.g., using a cross-correlation comparison) of theoretical intensities and observed intensity of MS2 fragment ions. If the neural score passes a defined threshold (e.g., indicating a sufficient match between the predicted and observed fragment ion intensities), a peptide is determined to be identified in the sample.

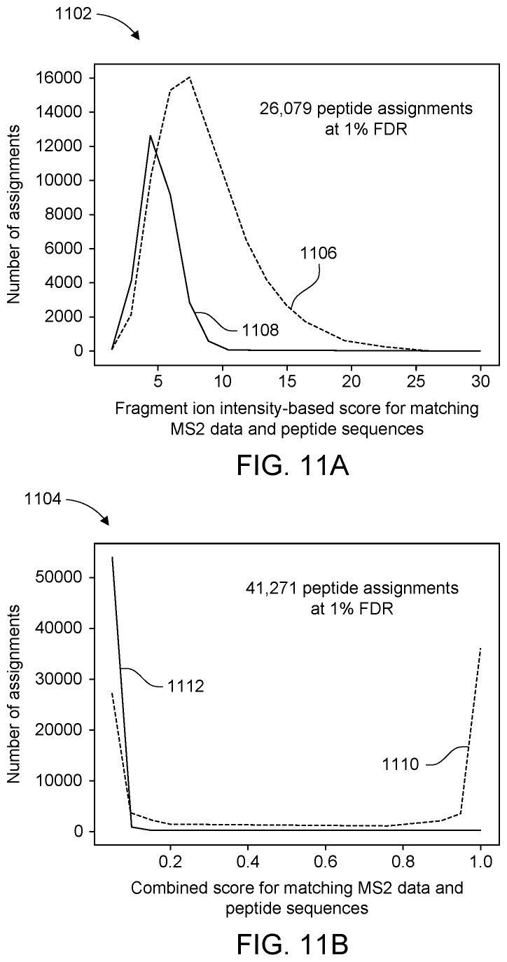

In some proteome mapping implementations, upon identifying that a peptide is present in the sample based on the neural score, one or more MS3 scans can be initiated for the corresponding fragment ions in order to perform quantification of the peptide. And once a defined number of peptides are quantified per protein, the peptide target list 214 can be updated to exclude any other peptides of the quantified protein. In other cases, however, calculating the neural score using the neural score module 228 can take too long, and a different approach can be implemented to determine, in real-time, which fragment ions to further subject to MS3 scans. For example, a pre-scoring process can be implemented by the MS2 validation module 238. Examples of pre-scoring processes are described in further detail below in relation to FIGS. 11A-11B.

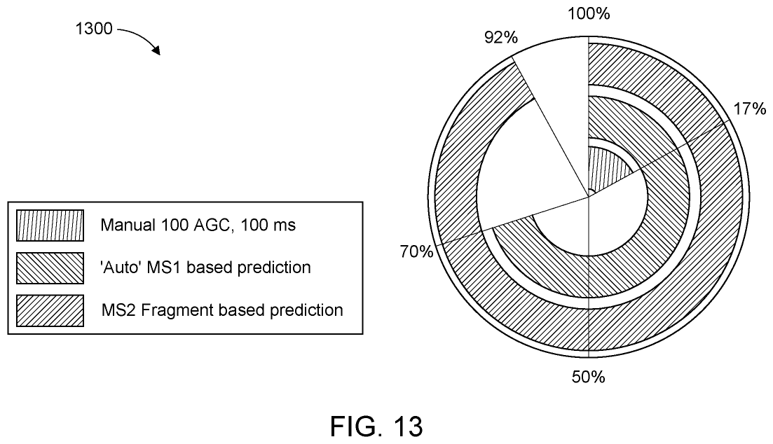

In addition to deciding which fragment ions to subject to MS3 scanning, the process 200 includes an MS3 accumulation prediction algorithm 240 that predicts (e.g., using MS3 accumulation prediction algorithm 240) an ion accumulation time that should be used for generating the MS3 spectra 210 in order to achieve a desired signal-to-noise ratio for peptide quantification. The MS3 accumulation prediction algorithm 240 and its performance is described in further detail below in relation to FIG. 13.

Using the process 200 (or variations of it), it is possible to reduce the time needed to map a plasma proteome by more than an order of magnitude compared to conventional high-throughput multiplexed MS approaches. For example, it has been shown that a variation of the process 200 has been able quantify more than 1,500 plasma proteins in 10 min. Thus, the proteome mapping processes described herein (e.g., the process 200) greatly enhance the use of mass spectrometry to identify biomarkers for early detection of cancer (or other diseases) from blood-plasma, and substantially increases the number of proteomes that can be mapped by a given MS system for a wide range of research activities.

Referring now to FIG. 3, a plot 300 is shown that illustrates a relationship between a number of peptide identifications by mass spectrometry and an intra-protein-observability (IPO) score (described above in relation to FIG. 2). The ultrahigh-throughput AI-driven mass spectrometry-based proteomics method described above (e.g., process 200) is a targeted method. The speed and sensitivity of the process 200 is based on identifying predefined peptides to cover a large portion of the analyzed proteome (optimally the entire proteome). In general, the process 200 allows the use of any and all proteins that are predicted to be encoded in a studied sample to be targeted. However, for practical applications, the method's sensitivity is increased by reducing the list of targeted peptides. Thus, it is desirable to identify the best peptides to be targeted for each protein in order to optimize the process 200. In some cases, the best peptides to be targeted for each protein can be identified by prior analyses (e.g., extensive off-line fractionation of a sample representing the studied samples and in-depth analysis of the samples using a DDA method). However, in other cases (e.g., in the process 200 shown in FIG. 2), the use of an observability module (e.g., observability module 212) can enable the prediction of peptides having the highest likelihood of being identified by the mass spectrometer. As described above, the metric used by the observability module 212 is referred to as the intra-protein-observability (IPO) score. The IPO score is based on measuring the variance of fragment intensities predicted by a neural network-based algorithm (e.g., the fragment prediction module 236). The smaller the variance across all possible fragment ions (corresponding to higher IPO scores), the higher is the likelihood that the peptide is identified by mass spectrometry. This relationship is confirmed by the data shown in the plot 300, generated by an analysis of a human blood plasma proteome sample. Thus, the IPO scores output by the observability module 212 can play an important role in selecting which target peptides to include in the peptide target list 214 to improve sensitivity of peptide identification when implementing the process 200.

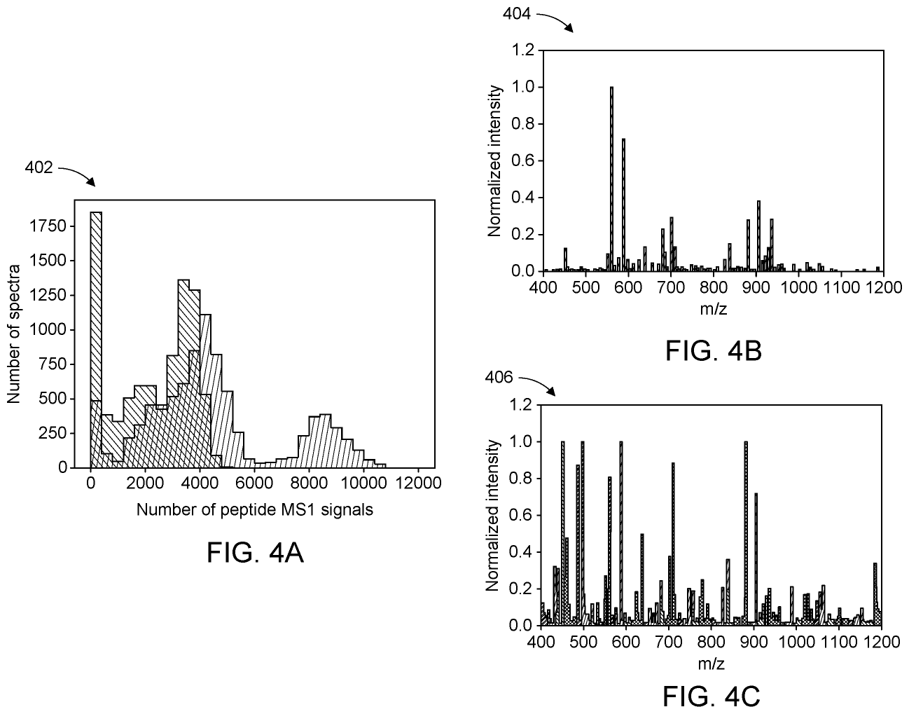

Referring to FIGS. 4A-4C, various plots 402, 404, 406 are shown to demonstrate the benefits of acquiring a MS1 spectrum (e.g., MS1 spectrum 206) in various sections compared to acquiring the MS1 spectrum using a single-section scan. For example, sectioning of the MS1 spectrum, as described herein, can be implemented by the MS1 calling algorithm 216 shown in FIG. 2 to optimize MS1 data acquisition to enhance sensitivity. FIG. 4A is a plot showing distributions of the number of peptide MS1 signals detected by (i) single-section MS1 spectra and (ii) 10-section MS1 spectra. FIG. 4B is an example spectrum from a single-section MS1 scan. FIG. 4C is an example spectrum from a 10-section MS1 scan.

In the process 200, a defined number of ions is analyzed for each MS1 spectrum acquired. The collection of the defined number of ions is achieved by varying the time during which ion collection is performed. Due to the finite number of ions that are collected, a small number of relatively high intensity ions dominating the ion mixture injected into the mass spectrometer 204 at any time during the measurement affects the ability to observe relatively low concentration peptides in the mixture. However, this issue can be overcome by running multiple individual MS1 spectra (e.g., 10 MS1, 20 MS1 spectra, etc.) across the mass-to-charge (m/z) range of interest and acquiring the same number of ions for each section. The ion packages of each section can then be combined in the mass spectrometer and analyzed together. Using this sectioning approach, if one section is dominated by a high intensity peptide ion signal, the ability of detecting low intensity signals in other sections will not be affected. The selected sections can be chosen (e.g., by the MS1 calling algorithm 216) based on several components including (i) the peptides in the peptide target list 214 predicted to elute at the given retention time and the given compensation voltage, and (ii) the signal in a given m/z region determined from previous MS1 scans (e.g., MS1 scans acquired during the same MS run at the same compensation voltage). In one implementation, m/z regions with high signals in previous MS1 scans are excluded, and the remaining m/z space is subdivided into multiple sections with comparable numbers of target peptides in each section. Plot 402 of FIG. 4A shows how the number of detectable MS1 peptide signals (with defined charges, 2-4) increases from 2.5 to 4 million when replacing a single-section MS1 scan with a 10-section MS1 scan, as described. Furthermore, comparing plot 404 of FIG. 4B (an MS1 scan acquired on a peptide ion mixture without sectioning the m/z space) and plot 406 of FIG. 4C (an MS1 scan acquired on the same peptide ion mixture but using a 10-section MS1 scan), it is observed that 10-section MS1 scan is much richer, with many more peptide signals detected.

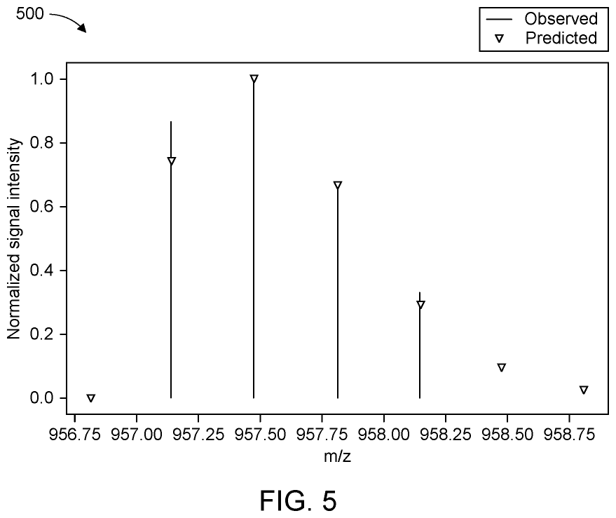

Referring now to FIG. 5, a plot 500 is shown, illustrating a comparison between observed and predicted signal intensities for a peptide ion. The observed peptide signal intensities correspond to peptide signal intensities for a peptide ion, as measured in an MS1 spectrum (e.g., MS1 spectrum 206). The predicted signal intensities correspond to predicted signal intensities, determined, for example, based on counting carbon atoms in the target peptide and calculating an envelope intensity distribution. Matching between predicted and observed MS1 intensities can be implemented in the peak detection module 222.

Peptide ion signals in mass spectrometry include a group of signals due to the natural distribution of isotopes of all elements included in the peptide. This isotope envelope is mainly driven by the natural occurrence of 13C (carbon-13). In the process 200, the MS1 signal envelope for each targeted peptide can therefore be predicted by counting the carbon atoms in the target peptide. These predictions can be matched with the signal envelopes of the measured data (e.g., by the peak detection module 222). Only if measured and predicted envelopes are determined to be a match (e.g., defined by a threshold score output by the peak detection module 222) is an MS2 spectrum acquired. This MS2 selection (e.g., performed by the MS2 caller module 224) includes a number of steps to refine the measured signal envelope and to determine the monoisotopic mass (e.g., of the peptide only made of the lightest isotopes of all elements) that is used for matching acquired MS2 data (e.g., MS2 spectra 208) with peptide sequences. These steps include: (a) calibrating the measured m/z values based on the mass spectrometer's current mass measurement accuracy deviation based on a number of identifications of selected high intensity peptide signals (e.g., peptides that are measured during the entire acquisition to adjust for mass deviations across the run); (b) averaging (e.g., weighted averaging) intensity signals across multiple MS1 spectra to enhance the signal distribution in the isotopic envelopes; (c) matching refined envelopes against envelopes predicted for targeted peptides based on their predicted retention time (e.g., predicted by the time module 218) and their predicted ion mobility compensation voltage (e.g., predicted by the CV module 220); and (d) determining matches of predicted and selected targeted signal envelopes using correlation measurements of signal intensities. Implementing these techniques yields good agreement between the measured and predicted signal intensities for a peptide ion, as shown in plot 500.

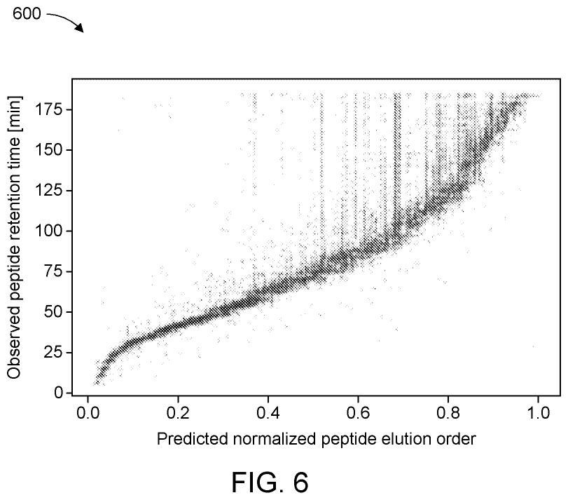

Referring now to FIG. 6, a plot 600 shows a relationship between observed peptide retention time and predicted peptide elution order for a plurality of peptides (e.g., with predictions being generated by the time module 218). Knowing the retention time of targeted peptides has two key functions in the process 200: (i) reducing the number of target peptides that are monitored for selection for MS2 at each given retention time, and (ii) improved validation of matches of MS2 spectra and peptide sequences. In some implementations, the time module 218 does not predict retention time directly, but instead predicts the order in which target peptides are eluted (which, in some cases, can be used to derive a predicted retention time). One advantage of predicting elution order rather than retention time is that the predictions are independent of the chromatographic system used (although currently developed only for reversed chromatography) as well as the gradient length used in the chromatography system. As described above, the time module 218 can be a neural network trained to predict elution order based on training data acquired from millions of previously recorded peptide elutions. The plot 600 shows good agreement between the measured elution times and predicted elution orders for a peptide mixture generated from a human plasma proteome sample. These results support the utility of the time module 218 used in the process 200

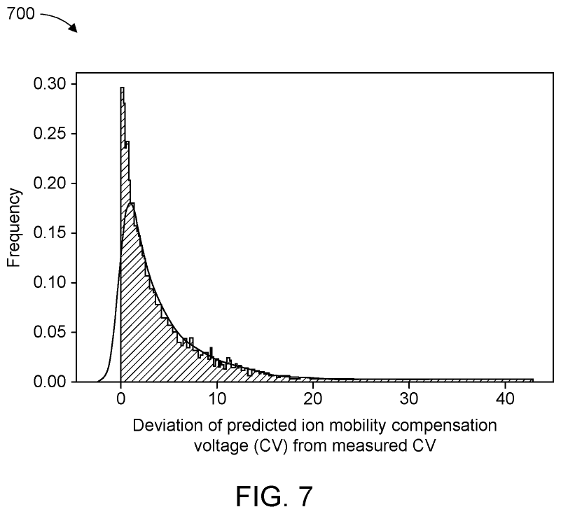

Referring now to FIG. 7, a plot 700 shows a distribution of the deviation between predicted and measured ion mobility compensation voltages (CV) (e.g., with the predicted CVs being generated by the CV module 220). Ion mobility is a technology that allows the fractionation of ions based on their structural topology (see, e.g., PMID: 23194268). This technology can be used to enable separated analysis of peptide ions that have identical m/z values and that elute at identical retention times. Ion mobility technology has also been shown to be especially advantageous when analyzing complex peptide mixtures such as those from plasma proteome samples (see, e.g., PMID: 33499602). In the process 200, an ion mobility device (e.g., a Thermo Fisher Scientific®, High-Field Asymmetric Waveform Ion Mobility Spectrometry (FAIMS) device [see, e.g., PMID: 30672687]) can be utilized to improve the throughput and/or sensitivity of proteome mapping. The fractionation of ions by FAIMS is achieved using different compensation voltages (CVs), and to optimize the process 200, the optimal CV setting for any peptide can be predicted using the CV module 220 (e.g., a neural network trained on CV data and MS spectra from previous MS runs). Similar to elution order (or retention time) prediction, the CV prediction helps to reduce the number of targeted peptides monitored at any given time during the analysis and supports the matching of acquired MS2 spectra with peptide sequences. The plot 700 shows a histogram of CV deviations between predicted and measured CVs for over 10,000 peptides. The median deviation is shown to be small (about 2 CVs) compared to the range of CVs covered in the process 200 (about 30 CVs). Thus, these results support the utility of the CV module 220 used in the process 200.

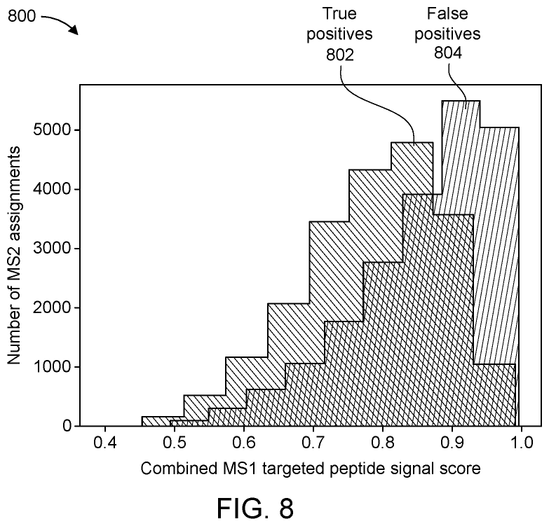

Referring now to FIG. 8, a plot 800 shows distributions of combined MS1 targeted peptide signal scores for a randomly selected sample of successful MS2 assignments (true positives 802) versus a randomly selected sample of unsuccessful MS2 assignments (false positives 804). The combined MS1 targeted peptide signal score refers to a metric that combines elution order (or retention time), mass accuracy of the observed mass (versus the true peptide mass), and CV prediction to determine a likelihood that the observed MS1 signal corresponds to a targeted peptide. In implementations of the process 200, an MS2 spectrum will only be acquired if the combined MS1 targeted peptide signal score exceeds a threshold value.

To produce the plot 800, the process 200 is implemented on a human plasma sample using, in combination, all of the above-described modules and algorithms that contribute to deciding in real-time if an MS2 spectrum is acquired for a detected MS1 peptide ion signal (e.g., observability module 212, MS1 calling algorithm 216, time module 218, CV module 220, peak detection module 222, and MS2 caller module 224). However, ultimately, it is the MS2 caller module 224 that makes the decision to acquire an MS2 spectrum for a detected peptide ion signal. Input values to the MS2 caller module 224 include: (i) the peptide target list 214 (including a likelihood of the peptide being observable by using mass spectrometry [e.g., as predicted by the observability module 212]), (ii) the MS1 peptide ion signal envelope, (iii) the deviation between observed and expected peptide ion mass (m/z), (iv) the peptide elution order [e.g., as predicted by the time module 218], and (v) the ion mobility compensation voltage [e.g., as predicted by the CV module 220]. The MS2 caller module 224 can decide to initiate an MS2 scan by processing these input values to generate a combined MS1 targeted peptide signal score, and then determining if the combined MS1 targeted peptide signal score satisfies a threshold condition. In some implementations of the MS2 caller module 224 (including the implementation used to produce the plot 800), all of the input values have to match defined thresholds for triggering MS2 data acquisition. Once MS2 scans are acquired, the match between MS2 data and target peptide sequences are then assessed and monitored in real time (e.g., using the neural score module 228 and/or the MS2 validation module 238) to identify the presence of target peptides in the sample. As previously described in relation to FIG. 2, once a defined number of peptides are identified and quantified for any protein (e.g., 3 peptides, 4 peptides, 5 peptides, etc.), all other target peptides for this protein are removed from the peptide target list 214. The peptide target list 214 can be modified in multiple ways to optimize the analysis including weighing of target peptides to focus on subgroups of peptides, weighing peptides based on the number of remaining peptides to still be eluted in the ongoing analysis, and deprioritizing (or prioritizing) the acquisition of MS1 peptide signals with high redundancy with respect to potential peptide sequence matches. Plot 800 shows separation between the successful MS2 assignments (e.g., true positives 802) and unsuccessful MS2 assignments (e.g., false positives 804), where the decision to acquire MS2 spectra was based solely on MS1 data. These results demonstrate that MS1 signals, by themselves, can be used in an efficient manner to avoid the acquisition of MS2 spectra that will not lead to the identification of target peptides.

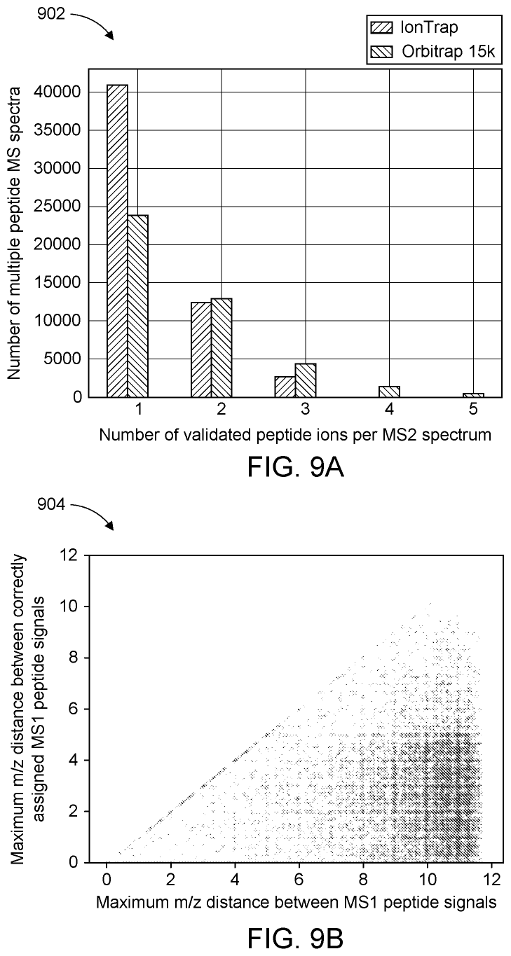

Referring now to FIGS. 9A-9B, plots 902 and 904 are shown to demonstrate the influence that resolution levels and isolation widths of acquired MS2 spectra can have on peptide identification (e.g., using the process 200). In FIG. 9A, plot 902 shows a number of successful peptide assignments from various 10 m/z isolation width MS2 spectra generated at two resolution levels. In FIG. 9B, plot 904 shows maximum m/z distances between correctly assigned MS1 peptide signals versus maximum m/z distances between MS1 peptide signals for various 10 m/z isolation width MS2 spectra.

In the process 200, the scoring for generating MS3 spectra for peptide quantification (e.g., performed by MS2 validation module 238) and the matching of MS2 data with peptide sequences (e.g., performed by the neural score module 228) allow for the identification of multiple peptides from a single MS2 spectrum. This is advantageous because MS2 spectra containing data from multiple peptide ions are commonly observed in proteomics mass spectrometry data from complex mixtures. The ability to identify multiple peptides from a single MS2 spectrum can therefore be leveraged enhance the overall speed of performing the process 200 for complex mixtures. To do this, the m/z window width for generating MS2 spectra can be optimized in real-time (e.g., by the wide window generator algorithm 226 of the MS2 caller module 224). The wide window generator algorithm 226 can perform widow width optimization based on (i) the intensity of peptide ion signal envelopes in the MS1 spectra, (ii) the number of peptide ion signals in a given m/z window, (iii) the MS1-based scoring for each peptide ion signal, (iv) expected ion accumulation for the MS2 spectra (e.g., as predicted based on the MS1 spectra), and/or (v) the ion accumulation time required for collecting sufficient numbers of ions for each individual peptide ion signal in the m/z window.

In FIG. 9A, plot 902 shows the number of successful peptide assignments from 10 m/z isolation width MS2 spectra generated at low resolution (the “IonTrap” bars) and 10 m/z isolation width MS2 spectra generated at high resolution (the “Orbitrap” bars) on a human cell line digest. These assignments (e.g., generated by the neural score module 228) show that high-resolution spectra enable the assignment of multiple precursors per spectra while low resolution spectra provide an enhanced peptide assignment for spectra dominated by one peptide ion. Thus, in some cases, the MS2 caller module 224 can be implemented to automatically select a mass spectrometry resolution setting in accordance with whether or not one expects multiple peptide ions to be present in the MS2 spectra (e.g., based on a complexity of the peptide mixture sample). High-resolution spectra acquisition typically requires more time that lower-resolution scans, but high-resolution spectra acquisition may still be advantageous for increasing proteome mapping throughput since it allows the simultaneous identification of more peptides from a single MS2 spectrum. Thus, for more complex samples where multiple peptide ions are expected to be present in a single MS2 spectrum, higher resolution levels may be preferred for MS2 spectra acquisition. Meanwhile, for simpler samples where only one dominant peptide ion is expected to be present in a single MS2 spectrum, lower resolution levels may be preferred.

In FIG. 9B, plot 904 shows that the majority of successfully assigned precursors from wide-window isolation MS2 spectra (10 m/z) are positioned within a sub-window having a width of less than 6 m/z. This data can help identify optimal isolation windows in real-time (e.g., using the wide window generator algorithm 226). Above a certain m/z window width, the MS2 spectrum may in some cases become noisy due to the high number of isolated peptide ions, making larger windows unattractive. However, in other cases, larger window sizes may result in more time-efficient identification of multiple peptide ions from a single MS2 spectrum. Thus, window width optimization (e.g., performed by the wide window generator algorithm 226) can be influential in increasing proteome mapping throughput.

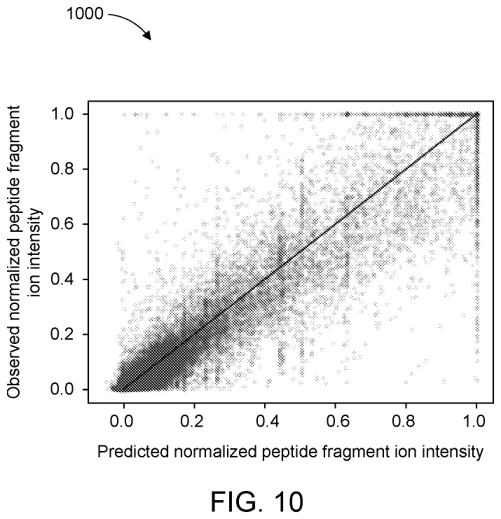

Referring now to FIG. 10, plot 1000 shows observed versus predicted normalized peptide fragment ion intensities for various peptide ions. The observed peptide fragment ion intensities were derived directly from acquired MS2 spectra (e.g., the MS2 spectra 208 shown in FIG. 2), while the predicted peptide fragment ion intensities were output by a fragment prediction module (e.g., the fragment prediction module 236 shown in FIG. 2). As described previously, the fragment prediction module 236 can be implemented using a neural network-based algorithm trained on previously captured MS2 spectra (e.g., thousands of training examples, hundreds of thousands of training examples, millions of training examples, etc.). In general, during MS2 scans, isolated peptide ions fragment along their amide backbone, and the amino acid sequences of the original peptides can then be determined based on the resulting fragment ions that are measured in the MS2 spectra. In the process 200, the fragment ion intensity predictions for the MS2 spectra (e.g., outputted by the fragment prediction module 236) are used to support the decision making of the acquisition of MS3 spectra for peptide quantification (e.g., decided by the MS2 validation module 238) and the final assignment of MS2 data to peptide amino acid sequences (e.g., assigned by the neural score module 228). The plot 1000 shows predicted and measured fragment ion intensities for one thousand peptide ions, demonstrating good agreement between the predictions and the measurements. In particular, the median Pearson correlation coefficient across all peptides was 0.97, confirming the utility of the fragment prediction module 236.

Referring now to FIGS. 11A-11B, plots 1102 and 1104 are shown to demonstrate the efficacy of using validation scores to decide on the acquisition of MS3 scans. In the process 200, peptide identification is mainly based on data from the acquired MS2 spectra (e.g., the MS2 spectra 208). For peptide quantification though, the process 200 includes the acquisition of MS3 spectra (e.g., the MS3 spectra 210) for multiple peptide fragment ions (e.g., peptide fragment ions selected from the MS2 spectra 208). It has been shown that MS3-based quantification substantially increases the accuracy of isobaric tag-based peptide quantification (see, e.g., PMID: 21963607, PMID: 24927332). However, the acquisition of MS3 spectra can be time consuming due to the high number of ions typically used for these spectra as well as the high-resolution data required for isobaric tag-based quantification. In the process 200, the final MS2-based peptide identification is performed using the neural score module 228, which can be implemented using a neural network-based algorithm. But, due to very short chromatographic peak widths of most peptides, decisions on generating a MS3 spectrum typically have to be made within a timeframe not compatible to using neural network-based algorithms. Therefore, instead of using the neural score module 228 to inform MS3 spectrum acquisition decisions, the process 200 applies a separate pre-scoring process (implemented by the MS2 validation module 238) to decide on the acquisition of MS3 scans. The prescoring (also referred to as “validation scoring”) can be based on one or more individual scores, including: (i) the correlation between predicted and observed fragment ion intensities, (ii) the deviation between predicted and observed retention time, (iii) the number of observed fragment ions relative to those predicted to be observed using the fragment prediction module 236, (iv) the mass accuracy of the observed MS1 peptide signal, and (v) a score reflecting the match between observed and predicted data based on a background-normalized dot-product (e.g., similar to tools/functions such as “XCorr” used in SEQUEST (described in PMID: 24226387) or Comet (described in PMID: 23148064)). In some implementations, the individual scores can be combined using a logistic regression model. The validation scoring process is also applicable in settings where multiple peptide amino acid sequences are assigned in one MS2 spectrum. For example, this includes settings where a portion of the fragment ion signals corresponding to a peptide sequence match are removed after identifying the match and before a new match with an additional peptide amino acid sequence is generated. The prediction of the fragment intensities (e.g., by the fragment prediction module 236) makes it possible to avoid unnecessarily removing all of a fragment signal, but rather removing only the portion assigned to the identified peptide. This enables identification of further peptides that may share specific fragment ions. In FIG. 11A, plot 1102 shows distributions of validation score values for true positive MS2 assignments (1106) and false positive MS2 assignments (1108) from a 3-hour mass spectrometry analysis of a human cell line proteome digest without any filtering of MS2 spectra performed based on the validation scores. In particular, the validation score metric used in plot 1102 corresponds to the above-mentioned score reflecting the match between observed and predicted data based on a background-normalized dot-product. In FIG. 11B, plot 1104 shows similar distributions of validation score values for true positive MS2 assignments (1110) and false positive MS2 assignments (1112) from the same 3-hour mass spectrometry analysis of a human cell line proteome digest. However, in plot 1104, the validation score metric used corresponds to the above-mentioned combined score using logistic regression. In both plots 1102 and 1104, false discovery rates of assignments were calculated using a target-decoy database approach (see, e.g., PMID: 17327847). Separation of true positive MS2 assignments and false positive MS2 assignments were observed in both plots 1102 and 1104, with much more pronounced separation shown in plot 1104 (41,271 peptide assignments at 1% false discovery rate versus 26,079 peptide assignments at 1% false discovery rate, respectively). These results demonstrate the efficacy of validation scoring for deciding on the acquisition of MS3 scans (especially the efficacy of implementing validation scoring using the above-mentioned combined score approach). By filtering MS2 spectra based on the validation scores described in this specification, higher numbers of true positive assignments can be achieved, while reducing the number of false positive assignments.

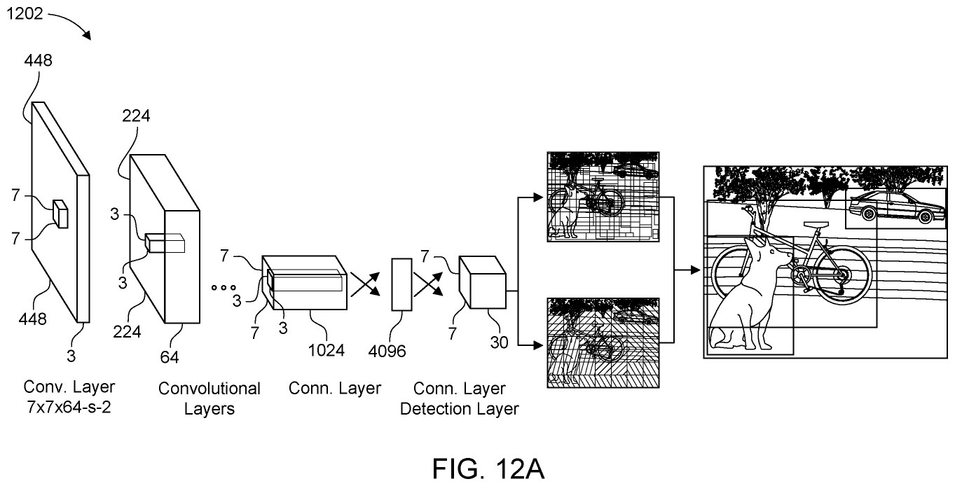

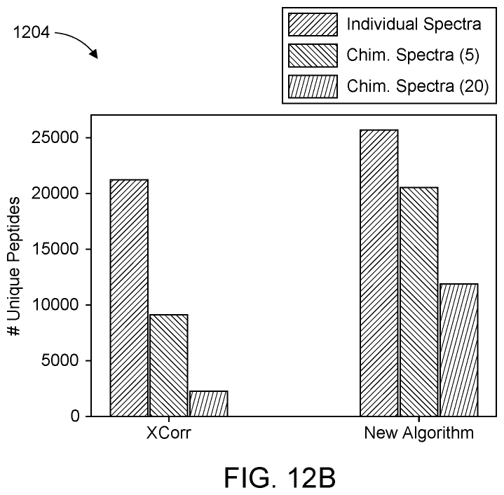

Referring now to FIGS. 12A-12B, it is described how MS2 data (e.g., the MS2 spectra 208) can be assigned to peptide amino acid sequences using a neural network-based algorithm (e.g., implemented as part of the neural score module 228). In some implementations, neural network-based assigning of MS2 spectra can be performed using one or more “You Only Look Once” YOLO-based image models (e.g., each trained on millions of examples of previously acquired MS2 spectra). These YOLO models can be fast enough to allow real-time scoring of peptide assignments (see, e.g., arxiv.org/abs/1506.02640 for a description of YOLO model for object detection). Referring briefly to FIG. 2, in the process 200, YOLO-based image models can be used to implement the predicted fragments module 230 and/or the observed fragments module 232. The general schema 1202 of a convolutional neural network (such as a YOLO model) is shown in FIG. 12A. In FIG. 12B, plot 1204 shows a comparison of results from assigning peptides to MS2 spectra using (i) an XCorr-scoring approach (denoted as “XCorr” along the horizontal axis) and (ii) a neural network-based approach (denoted as “New Algorithm” along the horizontal axis). For the assignment of multiple peptides to a single MS2 spectrum, fragment ion signals in the MS2 spectrum are only considered if they match predicted ion signals for a peptide amino acid sequence that corresponds to the MS1 peptide signal mass expected at the given retention time. In FIG. 12B, plot 1204 shows the results of the XCorr-scoring approach and the neural network-based approach for (i) a simulated dataset of spectra acquired for single peptides (e.g., the bars corresponding to “Individual Spectra”), (ii) a simulated dataset of spectra acquired for multiple peptide spectra generated through randomly mixing 5 spectra of individual single peptide MS2 data (e.g., the bars corresponding to “Chim. Spectra (5)”), and (iii) a simulated dataset of spectra acquired for multiple peptide spectra generated through randomly mixing 20 spectra of individual single peptide MS2 data (e.g., the bars corresponding to “Chim. Spectra (20)”). The results in plot 1204 show that the neural network-based approach generally outperforms the XCorr approach for spectra of all types. Specifically, while the SEQUEST XCorr approach only recovered less than half of the assignments for the 5-plexed spectra (and 10% of the assignments for the 20-plexed spectra), the neural network-based approach recovered more than 80% of the assignments for the 5-plexed spectra (and 46% of the assignments for the 20-plexed spectra). Thus, the implementation of the neural score module 228 using a neural network-based approach can yield substantial advantages compared to more conventional approaches for peptide assignments such as XCorr.

Referring now to FIG. 13, the plot 1300 compares signal-to-noise ratios achieved for peptide quantification using MS3 spectra obtained through various approaches. Accurate quantification using MS3 data can be achieved by the accumulation of enough peptide ions to reach a defined signal-to-noise threshold for the fragment ion signals (including isobaric tag reporter ion signals) measured in the MS3 spectra (e.g., MS3 spectra 210). In conventional methods implemented on mass spectrometers (e.g., Thermo Fisher Scientific® mass spectrometers), estimates of the ion accumulation time required to reach this threshold are based on the MS1 peptide ion signal intensity. However, for complex MS2 spectra comprising multiple peptide ions and for high noise spectra of low abundance peptides, the MS1 peptide ion signal intensity has limited predictive power, resulting in conventional methods producing spectra with undesirably low signal-to-noise characteristics. In the process 200, this issue is overcome by predicting the required ion accumulation time based on fragment ion intensities in the MS2 spectra (only considering fragment ions of individual peptides rather than all of the fragment ions observed in an MS2 spectrum of multiple peptide ions). For example, this prediction can be performed by the MS3 accumulation prediction algorithm 240, which can be implemented as a generalized random forest model trained on previous examples of MS3 spectra and their associated ion accumulation times. The plot 1300 shows, for each of three methods, the percentage of MS3 spectra acquired that provide peptide quantification with a signal-to-noise ratio greater than ten. For the “MS2 Fragment based prediction” implemented by the MS3 accumulation prediction algorithm 240, this percentage was 92%. This was substantially larger than the percentages yielded by two standard methods provided by a Thermo Fisher Scientific® mass spectrometer—an “‘Auto’ MS1 based prediction” and a “Manual 100 AGC, 100 ms” setting—which respectively yielded percentages of 70% and 17%. These results support the utility of the MS3 accumulation prediction algorithm 240 and demonstrate its superior performance compared to conventional approaches to acquiring MS3 spectra.

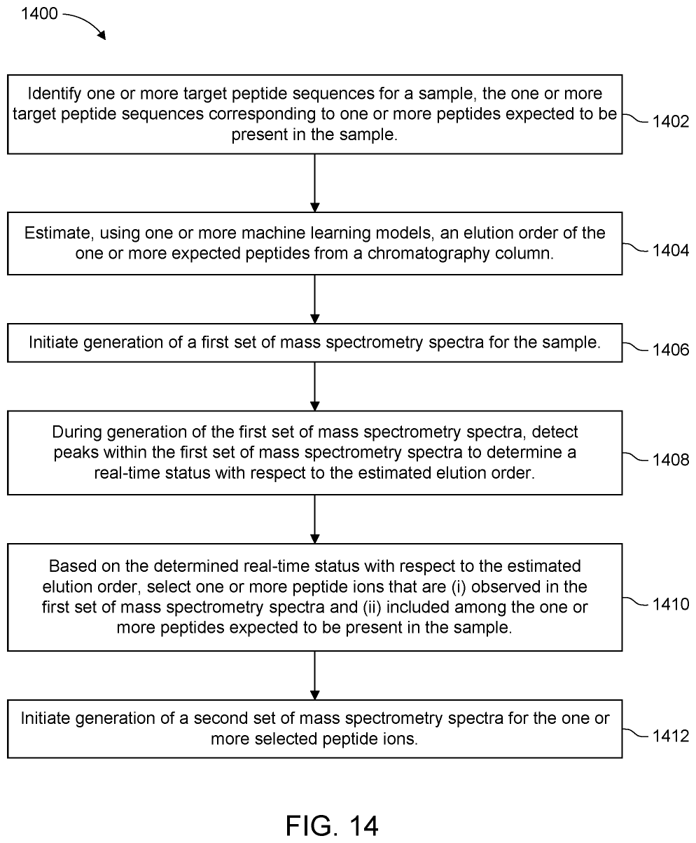

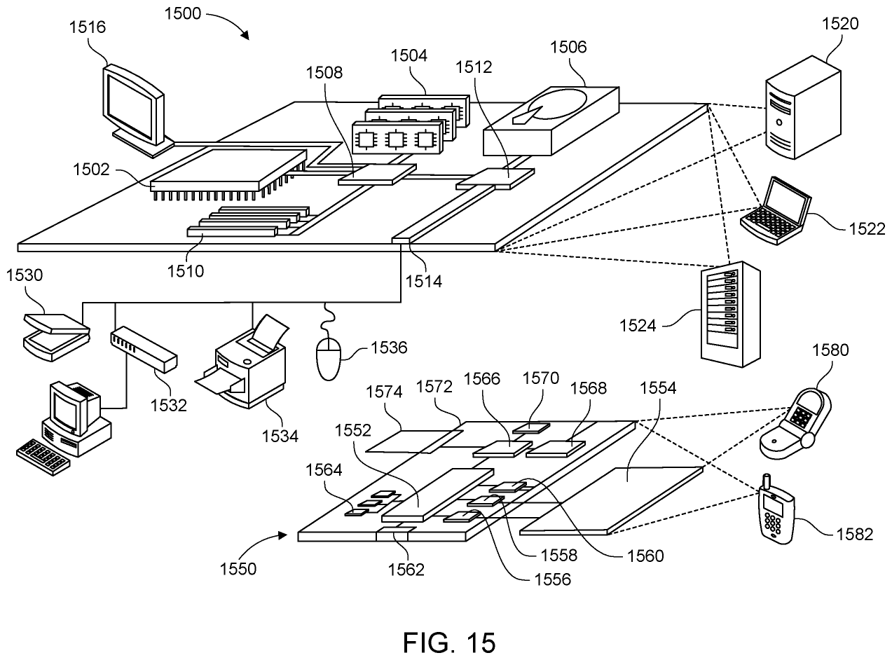

FIG. 14 illustrates an example process 1400 for proteome mapping. In some implementations, operations of the process 1400 can be executed by a computing device or mobile computing device such as those described below in relation to FIG. 15. Operations of the process 1400 include identifying one or more target peptide sequences for a sample, the one or more target peptide sequences corresponding to one or more peptides expected to be present in the sample (1402). In some implementations, the one or more target peptide sequences can correspond to one or more peptides on a peptide target list (e.g., the peptide target list 214). Identifying the one or more target peptide sequences for the sample can include: predicting, using one or more additional machine learning models (e.g., the fragment prediction module 236), fragment intensities of mass spectrometry spectra of a plurality of peptides; ranking the plurality of peptides based on a metric indicative of a variance of the predicted fragment intensities for each of the plurality peptides; and selecting (e.g., using the observability module 212) a subset of the plurality of peptides that has the lowest values of the metric. In some implementations, the sample can be an unfractionated sample and/or a sample that is chemically tagged with an isobaric mass tag.

Operations of the process 1400 also include estimating, using one or more machine learning models, an elution order of the one or more expected peptides from a chromatography column (1404). In some implementations, the one or machine learning models can correspond to, e.g., a neural network algorithm implemented as part of the time module 218 described above.

Operations of the process 1400 also include initiating generation of a first set of mass spectrometry spectra for the sample (1406). In some implementations, the first set of mass spectrometry spectra can correspond to MS1 spectra (e.g., the MS1 spectrum 206) and initiating generation of the first set of mass spectrometry spectra can correspond to a function performed by the MS1 calling algorithm 216 described above. In some implementations, initiating generation of the first set of mass spectrometry spectra for the sample can include generating a plurality of individual spectra having different mass-to-charge ranges (e.g., the sectioning of MS1 spectra described above in relation to FIGS. 4A-4C). The different mass-to-charge ranges can be selected based on at least one of (i) the one or more target peptide sequences, (ii) the determined real-time status with respect to the estimated elution order, (iii) intensities of previously recorded signals in the given mass-to-charge ranges, or (iv) compensation voltage predictions.

Operations of the process 1400 also include, during generation of the first set of mass spectrometry spectra, detecting peaks within the first set of mass spectrometry spectra to determine a real-time status with respect to the estimated elution order (1408). In some implementations, step 1408 can correspond to a function performed by the peak detection algorithm 222 described above.

Operations of the process 1400 also include, based on the determined real-time status with respect to the estimated elution order, selecting one or more peptide ions that are (i) observed in the first set of mass spectrometry spectra and (ii) included among the one or more peptides expected to be present in the sample (1410). In some implementations, step 1410 can correspond to a function performed by the MS2 caller module 224 described above.

Operations of the process 1400 also include initiating generation of a second set of mass spectrometry spectra for the one or more selected peptide ions (1412). In some implementations, the second set of mass spectrometry spectra can correspond to the MS2 spectra 208, and step 1412 can correspond to a function performed by the MS2 caller module 224 described above. In some implementations, initiating generation of the second set of mass spectrometry spectra for the one or more selected peptide ions can include defining a width of a mass-to-charge range for at least one spectrum of the second set of mass spectrometry spectra, the width being defined based on (i) intensities of signals in the first set of mass spectrometry spectra, (ii) a number of peptide ion signals in a given mass-to-charge range, and (iii) an estimated accumulation time required for collecting a threshold number of ions for each of the peptide ion signals in the given mass-to-charge range. For example, defining the width of the mass-to-charge range can correspond to a function performed by the wide window generator algorithm 226 described above. In some implementations, initiating generation of the second set of mass spectrometry spectra for the one or more selected peptide ions can include isolating the one or more selected peptide ions in a mass spectrometer that produces the mass spectrometry spectra (e.g., the mass spectrometer 204), fragmenting the one or more selected peptide ions to generate fragment ions, and recording measurements related to at least a portion of the generated fragment ions.

Additional operations of the process 1400 can include the following. In some implementations, the process 1400 can include estimating one or more ion mobility properties of peptide ions that maximize sensitivity of a mass spectrometer to the peptide ions. The one or more ion mobility properties of the peptide ions that maximize sensitivity of the mass spectrometer to the peptide ions can include a compensation voltage (e.g., estimated using the CV module 220), and selecting the one or more peptide ions that are (i) observed in the first set of mass spectrometry spectra and (ii) included among the one or more peptides expected to be present in the sample can be additionally based on the compensation voltage.

In some implementations, the process 1400 can include analyzing the second set of mass spectrometry spectra, wherein the analyzing includes inputting data indicative of the second set of mass spectrometry spectra into one or more convolutional neural networks trained to identify a presence of one or more peptides in the sample based on the data indicative of the second set of mass spectrometry spectra. For example, the one or more convolutional neural networks can correspond to the predicted fragments module 230 and the observed fragments module 232 of the neural score module 228 described above.

In some implementations, the process 1400 can include selecting one or more fragment ions that are observed in the second set of mass spectrometry spectra; and initiating generation of a third set of mass spectrometry spectra for the one or more selected fragment ions. For example, selecting the one or more fragment ions can correspond to a function performed by the MS2 caller module 224 described above, and the third set of mass spectrometry spectra can be MS3 spectra such as the MS3 spectra 210 described above. In some implementations, the third set of mass spectrometry spectra can be generated by (i) isolating the one or more selected fragment ions, (ii) further fragmenting the one or more selected fragment ions to produce further fragmented ions, and (iii) detecting at least a portion of the further fragmented ions, wherein the further fragmented ions include isobaric tag reporter ions. In some implementations, selecting the one or more fragment ions that are observed in the second set of mass spectrometry spectra can include scoring the one or more fragment ions based on at least one of: (i) a correlation between predicted and observed fragment ion intensities, (ii) a deviation between predicted and observed retention times for the one or more expected peptides, (iii) a number of observed fragment ions relative to a number of fragment ions predicted to be observed, (iv) a mass accuracy of an observed peptide signal from the first set of mass spectrometry spectra, and (v) a score reflecting a match between observed and predicted data based on a background-normalized dot-product. In some implementations, initiating the generation of the third set of mass spectrometry spectra for the one or more selected fragment ions can include estimating a time required for collecting a threshold amount of each of the one or more selected fragment ions that correspond to a single peptide, the threshold amount corresponding to a signal-to-noise threshold for isobaric tag reporter ion signals; and initiating the generation of the third set of mass spectrometry spectra to collect data for at least the estimated time. For example, the estimated time can be an ion accumulation time, and the estimation can correspond to a function performed by the MS3 accumulation prediction algorithm 240 described above. In some implementations, the process 1400 can include analyzing the third set of mass spectrometry spectra for the one or more selected fragment ions to quantify an amount of at least one detected peptide present in the sample.

In some implementations, the process 1400 can include monitoring a mass-to-charge ratio of intact peptide ions in the first set of mass spectrometry spectra.

FIG. 15 shows an example of a computing device 1500 and a mobile computing device 1550 that are employed to execute implementations of the present disclosure. For example, the computing device 1500 and/or the mobile computing device 1550 can be employed (e.g., through the execution of computer readable instructions) to implement one or more of the modules and/or algorithms shown in FIG. 2 such as the observability module 212, the MS1 calling algorithm 216, the time module 218, the CV module 220, the peak detection algorithm 222, the MS2 caller module 224, the wide window generator algorithm 226, the neural score module 228, the predicted fragments module 230, the observed fragments module 232, the fragment prediction module 236, the MS2 validation module 238, and/or the MS3 accumulation prediction algorithm 240. The computing device 1500 and/or mobile computing device can also be employed to execute the process 1400, including one or more of its constituent operations such as operations 1402, 1404, 1406, 1408, 1410, and 1412. In some implementations, multiple computing devices (e.g., multiple computing devices 1500, multiple mobile computing device 1550, or some combination of computing devices 1500 and mobile computing devices 1550)—located either locally or remotely—can be employed to accomplish the same ends. For example, the multiple computing devices and/or mobile computing devices can be connected to one another on the same local network, or via the cloud.

The computing device 1500 is intended to represent various forms of digital computers, such as laptops, desktops, workstations, personal digital assistants, servers, blade servers, mainframes, and other appropriate computers. The mobile computing device 1550 is intended to represent various forms of mobile devices, such as personal digital assistants, cellular telephones, smart-phones, AR devices, and other similar computing devices. The components shown here, their connections and relationships, and their functions, are meant to be examples only, and are not meant to be limiting. In some implementations of the technology disclosed herein, the computing device 1500 and/or the mobile computing device 1550 can correspond to a device embedded or communicably connected to a mass spectrometer (e.g., the mass spectrometer 204) and can cause the mass spectrometer to perform one or more operations.

The computing device 1500 includes a processor 1502, a memory 1504, a storage device 1506, a high-speed interface 1508, and a low-speed interface 1512. In some implementations, the high-speed interface 1508 connects to the memory 1504 and multiple high-speed expansion ports 1510. In some implementations, the low-speed interface 1512 connects to a low-speed expansion port 1514 and the storage device 1504. Each of the processor 1502, the memory 1504, the storage device 1506, the high-speed interface 1508, the high-speed expansion ports 1510, and the low-speed interface 1512, are interconnected using various buses, and may be mounted on a common motherboard or in other manners as appropriate. The processor 1502 can process instructions for execution within the computing device 1500, including instructions stored in the memory 1504 and/or on the storage device 1506 to display graphical information for a graphical user interface (GUI) on an external input/output device, such as a display 1516 coupled to the high-speed interface 1508. In other implementations, multiple processors and/or multiple buses may be used, as appropriate, along with multiple memories and types of memory. In addition, multiple computing devices may be connected, with each device providing portions of the necessary operations (e.g., as a server bank, a group of blade servers, or a multi-processor system).

The memory 1504 stores information within the computing device 1500. In some implementations, the memory 1504 is a volatile memory unit or units. In some implementations, the memory 1504 is a non-volatile memory unit or units. The memory 1504 may also be another form of a computer-readable medium, such as a magnetic or optical disk.

The storage device 1506 is capable of providing mass storage for the computing device 1500. In some implementations, the storage device 1506 may be or include a computer-readable medium, such as a floppy disk device, a hard disk device, an optical disk device, a tape device, a flash memory, or other similar solid-state memory device, or an array of devices, including devices in a storage area network or other configurations. Instructions can be stored in an information carrier. The instructions, when executed by one or more processing devices, such as processor 1502, perform one or more methods, such as those described above. The instructions can also be stored by one or more storage devices, such as computer-readable or machine-readable mediums, such as the memory 1504, the storage device 1506, or memory on the processor 1502.

The high-speed interface 1508 manages bandwidth-intensive operations for the computing device 1500, while the low-speed interface 1512 manages lower bandwidth-intensive operations. Such allocation of functions is an example only. In some implementations, the high-speed interface 1508 is coupled to the memory 1504, the display 1516 (e.g., through a graphics processor or accelerator), and to the high-speed expansion ports 1510, which may accept various expansion cards. In the implementation, the low-speed interface 1512 is coupled to the storage device 1506 and the low-speed expansion port 1514. The low-speed expansion port 1514, which may include various communication ports (e.g., Universal Serial Bus (USB), Bluetooth, Ethernet, wireless Ethernet) may be coupled to one or more input/output devices. Such input/output devices may include a scanner, a printing device, or a keyboard or mouse. The input/output devices may also be coupled to the low-speed expansion port 1514 through a network adapter. Such network input/output devices may include, for example, a switch or router.

The computing device 1500 may be implemented in a number of different forms, as shown in FIG. 15. For example, it may be implemented as a standard server 1520, or multiple times in a group of such servers. In addition, it may be implemented in a personal computer such as a laptop computer 1522. It may also be implemented as part of a rack server system 1524. Alternatively, components from the computing device 1500 may be combined with other components in a mobile device, such as a mobile computing device 1550. Each of such devices may contain one or more of the computing device 1500 and the mobile computing device 1550, and an entire system may be made up of multiple computing devices communicating with each other.

The mobile computing device 1550 includes a processor 1552; a memory 1564; an input/output device, such as a display 1554; a communication interface 1566; and a transceiver 1568; among other components. The mobile computing device 1550 may also be provided with a storage device, such as a micro-drive or other device, to provide additional storage. Each of the processor 1552, the memory 1564, the display 1554, the communication interface 1566, and the transceiver 1568, are interconnected using various buses, and several of the components may be mounted on a common motherboard or in other manners as appropriate. In some implementations, the mobile computing device 1550 may include a camera device(s).

The processor 1552 can execute instructions within the mobile computing device 1550, including instructions stored in the memory 1564. The processor 1552 may be implemented as a chipset of chips that include separate and multiple analog and digital processors. For example, the processor 1552 may be a Complex Instruction Set Computers (CISC) processor, a Reduced Instruction Set Computer (RISC) processor, or a Minimal Instruction Set Computer (MISC) processor. The processor 1552 may provide, for example, for coordination of the other components of the mobile computing device 1550, such as control of user interfaces (UIs), applications run by the mobile computing device 1550, and/or wireless communication by the mobile computing device 1550.

The processor 1552 may communicate with a user through a control interface 1558 and a display interface 1556 coupled to the display 1554. The display 1554 may be, for example, a Thin-Film-Transistor Liquid Crystal Display (TFT) display, an Organic Light Emitting Diode (OLED) display, or other appropriate display technology. The display interface 1556 may include appropriate circuitry for driving the display 1554 to present graphical and other information to a user. The control interface 1558 may receive commands from a user and convert them for submission to the processor 1552. In addition, an external interface 1562 may provide communication with the processor 1552, so as to enable near area communication of the mobile computing device 1550 with other devices. The external interface 1562 may provide, for example, for wired communication in some implementations, or for wireless communication in other implementations, and multiple interfaces may also be used.

The memory 1564 stores information within the mobile computing device 1550. The memory 1564 can be implemented as one or more of a computer-readable medium or media, a volatile memory unit or units, or a non-volatile memory unit or units. An expansion memory 1574 may also be provided and connected to the mobile computing device 1550 through an expansion interface 1572, which may include, for example, a Single in Line Memory Module (SIMM) card interface. The expansion memory 1574 may provide extra storage space for the mobile computing device 1550, or may also store applications or other information for the mobile computing device 1550. Specifically, the expansion memory 1574 may include instructions to carry out or supplement the processes described above, and may include secure information also. Thus, for example, the expansion memory 1574 may be provided as a security module for the mobile computing device 1550, and may be programmed with instructions that permit secure use of the mobile computing device 1550.

In addition, secure applications may be provided via the SIMM cards, along with additional information, such as placing identifying information on the SIMM card in a non-hackable manner.

The memory may include, for example, flash memory and/or non-volatile random access memory (NVRAM), as discussed below. In some implementations, instructions are stored in an information carrier. The instructions, when executed by one or more processing devices, such as processor 1552, perform one or more methods, such as those described above. The instructions can also be stored by one or more storage devices, such as one or more computer-readable or machine-readable mediums, such as the memory 1564, the expansion memory 1574, or memory on the processor 1552. In some implementations, the instructions can be received in a propagated signal, such as, over the transceiver 1568 or the external interface 1562.

The mobile computing device 1550 may communicate wirelessly through the communication interface 1566, which may include digital signal processing circuitry where necessary. The communication interface 1566 may provide for communications under various modes or protocols, such as Global System for Mobile communications (GSM) voice calls, Short Message Service (SMS), Enhanced Messaging Service (EMS), Multimedia Messaging Service (MMS) messaging, code division multiple access (CDMA), time division multiple access (TDMA), Personal Digital Cellular (PDC), Wideband Code Division

Multiple Access (WCDMA), CDMA2000, General Packet Radio Service (GPRS). Such communication may occur, for example, through the transceiver 1568 using a radio frequency. In addition, short-range communication, such as using a Bluetooth or Wi-Fi, may occur. In addition, a Global Positioning System (GPS) receiver module 1570 may provide additional navigation- and location-related wireless data to the mobile computing device 1550, which may be used as appropriate by applications running on the mobile computing device 1550.

The mobile computing device 1550 may also communicate audibly using an audio codec 1560, which may receive spoken information from a user and convert it to usable digital information. The audio codec 1560 may likewise generate audible sound for a user, such as through a speaker, e.g., in a handset of the mobile computing device 1550. Such sound may include sound from voice telephone calls, may include recorded sound (e.g., voice messages, music files, etc.) and may also include sound generated by applications operating on the mobile computing device 1550.

The mobile computing device 1550 may be implemented in a number of different forms, as shown in FIG. 15. For example, it may be implemented a phone device 1580, a personal digital assistant 1582, and a tablet device (not shown). The mobile computing device 1550 may also be implemented as a component of a smart-phone, AR device, or other similar mobile device.

Computing device 1500 and/or 1550 can also include USB flash drives. The USB flash drives may store operating systems and other applications. The USB flash drives can include input/output components, such as a wireless transmitter or USB connector that may be inserted into a USB port of another computing device.

Other embodiments and applications not specifically described herein are also within the scope of the following claims. Elements of different implementations described herein may be combined to form other embodiments not specifically set forth above. Elements may be left out of the structures described herein without adversely affecting their operation. Furthermore, various separate elements may be combined into one or more individual elements to perform the functions described herein.

### EXAMPLES

In one example, three peptides can be selected as targets for each protein and the recorded information for the peptides can be commonly detected MS2 fragment ions and nano-capillary HPLC retention time. A possible aim of the example could be to quantitatively map all 6000 target peptides from 2000 target proteins in an unfractionated TMT labeled plasma digest. The difficulty of this is that many of the peptide ions will have a full-MS intensity that does not exceed the noise level. These peptides can be accurately quantified based on known fragment ions using an MS3 experiment. However, this is only possible if it is known at which nano-capillary retention time the peptide is eluted into the mass spectrometer. Other groups have solved this problem by spiking synthetic forms of peptides into the sample, using these synthetic forms of peptides as pilot peptides to determine the exact retention time of the target peptides. However, the number of 6000 targets is at the upper limit of the capacity of this approach, and the costs of synthesizing 6000 peptides can counter the cost-reduction of the analysis through higher-throughput proteome mapping. The technologies disclosed in this specification can overcome this problem by predicting the exact retention times of peptides using peptides with signal intensity above the noise level as internal standards. One aim, using the technologies disclosed herein, is the quantification of all 2000 proteins from unfractionated TMT labeled plasma proteome digests. This would represent a sample throughput increase of more than 10-fold compared to the current method. The technologies disclosed in this specification have the potential to allow mapping more than 70 plasma proteome samples to a depth of 2000 proteins in 24 hours on one mass spectrometer and lowering the overall cost for mapping one plasma proteome by at least 2-fold.

### OTHER EMBODIMENTS

It is to be understood that while the invention has been described in conjunction with the detailed description thereof, the foregoing description is intended to illustrate and not limit the scope of the invention, which is defined by the scope of the appended claims. The following are numbered embodiments intended to further illustrate, but not limit, the scope of the invention.
1. A method for high-throughput mapping of proteomes from a proteome sample of a subject, comprising: obtaining a proteome sample from said subject, wherein said sample comprises microparticles including peptides; performing a targeted mass spec analysis of said sample using a real-time predictor of peptide retention times to generate targeted proteomic data; and mapping said targeted proteomic data based on one or more features of said proteomic data, wherein said one or more features are indicative of one or more biomarker.
2. The method of embodiment 1, further comprising generating a phenotype classification based on said one or more biomarker.
3. The method of embodiment 2, wherein said one or more phenotype classifications are selected from the group consisting of: a drug response state, a disease state, a non-disease state, and any combination thereof.
4. The method of embodiment 1, wherein the real-time predictor comprises an artificial intelligence model trained to identify pre-eluding peptides and predict exact retention times of the peptides essential to assessing the one or more features.

### REFERENCES

PMID: 12713048
Thompson A, Schäfer J, Kuhn K, Kienle S, Schwarz J, Schmidt G, Neumann T, Johnstone R, Mohammed A K, Hamon C. Tandem mass tags: a novel quantification strategy for comparative analysis of complex protein mixtures by MS/MS. Anal Chem. 2003 Apr. 15; 75(8):1895-904. doi: 10.1021/ac0262560. Erratum in: Anal Chem. 2003 Sep. 15; 75(18):4942. Johnstone, R [added]. Erratum in: Anal Chem. 2006 Jun. 15; 78(12):4235. Mohammed, A Karim A [added]. PMID: 12713048.
PMID: 21963607
Ting L, Rad R, Gygi S P, Haas W. MS3 eliminates ratio distortion in isobaric multiplexed quantitative proteomics. Nat Methods. 2011 Oct. 2; 8(11):937-40. doi: 10.1038/nmeth. 1714. PMID: 21963607
PMID: 24927332
McAlister G C, Nusinow D P, Jedrychowski M P, Wühr M, Huttlin E L, Erickson B K, Rad R, Haas W, Gygi S P. MultiNotch MS3 enables accurate, sensitive, and multiplexed detection of differential expression across cancer cell line proteomes. Anal Chem. 2014 Jul. 15; 86(14):7150-8. doi: 10.1021/ac502040v. Epub 2014 Jul. 3. PMID: 24927332.
PMID: 32203386
Li J, Van Vranken J G, Pontano Vaites L, Schweppe D K, Huttlin E L, Etienne C, Nandhikonda P, Viner R, Robitaille A M, Thompson A H, Kuhn K, Pike I, Bomgarden R D, Rogers J C, Gygi SP, Paulo J A. TMTpro reagents: a set of isobaric labeling mass tags enables simultaneous proteome-wide measurements across 16 samples. Nat Methods. 2020 April; 17(4): 399-404. doi: 10.1038/s41592-020-0781-4. Epub 2020 Mar. 16. PMID: 32203386.
PMID: 15385600
Ross P L, Huang Y N, Marchese J N, Williamson B, Parker K, Hattan S, Khainovski N, Pillai S, Dey S, Daniels S, Purkayastha S, Juhasz P, Martin S, Bartlet-Jones M, He F, Jacobson A, Pappin D J. Multiplexed protein quantitation in Saccharomyces cerevisiae using amine-reactive isobaric tagging reagents. Mol Cell Proteomics. 2004 December; 3(12):1154-69. doi: 10.1074/mcp.M400129-MCP200. Epub 2004 Sep. 22. PMID: 15385600.
PMID: 33900084
Li J, Cai Z, Bomgarden R D, Pike I, Kuhn K, Rogers J C, Roberts T M, Gygi S P, Paulo J A. TMTpro-18plex. The Expanded and Complete Set of TMTpro Reagents for Sample Multiplexing. J Proteome Res. 2021 May 7; 20(5):2964-2972. doi: 10.1021/acs.jproteome.1c00168. Epub 2021 Apr. 26. PMID: 33900084.
PMID: 28938075
Schwenk J M, Omenn G S, Sun Z, Campbell D S, Baker M S, Overall C M, Aebersold R, Moritz R L, Deutsch E W. The Human Plasma Proteome Draft of 2017: Building on the Human Plasma PeptideAtlas from Mass Spectrometry and Complementary Assays. J Proteome Res. 2017 Dec. 1: 16(12):4299-4310. doi: 10.1021/acs.jproteome.7b00467. Epub 2017 Oct. 10. PMID: 28938075.
PMID: 28065596
Erickson B K, Rose C M, Braun C R, Erickson A R, Knott J, McAlister G C, Wühr M, Paulo J A, Everley R A, Gygi S P. A Strategy to Combine Sample Multiplexing with Targeted Proteomics Assays for High-Throughput Protein Signature Characterization. Mol Cell. 2017 Jan. 19; 65(2):361-370. doi: 10.1016/j.molcel.2016.12.005. Epub 2017 Jan. 5. PMID: 28065596.
PMID: 32332170
Yu Q. Xiao H, Jedrychowski M P, Schweppe D K, Navarrete-Perea J, Knott J, Rogers J, Chouchani E T, Gygi S P. Sample multiplexing for targeted pathway proteomics in aging mice. Proc Natl Acad Sci USA. 2020 May 5; 117(18):9723-9732. doi: 10.1073/pnas.1919410117. Epub 2020 Apr. 24. PMID: 32332170.
PMID: 30672687
Schweppe D K, Prasad S, Belford M W, Navarrete-Perea J, Bailey D J, Huguet R, Jedrychowski M P, Rad R, McAlister G, Abbatiello S E, Woulters E R, Zabrouskov V, Dunyach J J, Paulo J A, Gygi S P. Characterization and Optimization of Multiplexed Quantitative Analyses Using High-Field Asymmetric-Waveform Ion Mobility Mass Spectrometry. Anal Chem. 2019 Mar. 19, 91(6):4010-4016. doi: 10.1021/acs.analchem.8b05399. Epub 2019 Feb. 26. Erratum in: Anal Chem. 2020 Mar. 17; 92(6):4690. PMID: 30672687.
PMID: 23194268
Swearingen K E, Moritz R L. High-field asymmetric waveform ion mobility spectrometry for mass spectrometry-based proteomics. Expert Rev Proteomics. 2012 October; 9(5):505-17. doi: 10.1586/epr.12.50. PMID: 23194268.
PMID: 33499602
Gaun A, Lewis Hardell K N, Olsson N, O'Brien J J, Gollapudi S, Smith M, McAlister G, Huguet R, Keyser R, Buffenstein R, McAllister F E. Automated 16-Plex Plasma Proteomics with Real-Time Search and Ion Mobility Mass Spectrometry Enables Large-Scale Profiling in Naked Mole-Rats and Mice. J Proteome Res. 2021 Feb. 5; 20(2):1280-1295. doi: 10.1021/acs.jproteome.0c00681. Epub 2021 Jan. 26. PMID: 33499602.
PMID: 24226387
Eng J K, McCormack A L, Yates J R. An approach to correlate tandem mass spectral data of peptides with amino acid sequences in a protein database. J Am Soc Mass Spectrom. 1994 November; 5(11):976-89. doi: 10.1016/1044-0305(94)80016-2. PMID: 24226387.
PMID: 23148064
Eng J K, Jahan T A, Hoopmann M R. Comet: an open-source MS/MS sequence database search tool. Proteomics. 2013 January; 13(1):22-4. doi: 10.1002/pmic.201200439. Epub 2012 Dec. 4. PMID: 23148064.
PMID: 17327847
Elias J E, Gygi S P. Target-decoy search strategy for increased confidence in large-scale protein identifications by mass spectrometry. Nat Methods. 2007 March; 4(3):207-14. doi: 10.1038/nmeth1019. PMID: 17327847.
arxiv.org/abs/1506.02640
Redmon J, Divvala S, Ross G, Farhadi A. You Only Look Once: Unified, Real-Time Object Detection. arXiv. 2016 May. arXiv: 1506.02640v5.

1. A method comprising:
identifying one or more target peptide sequences for a sample, the one or more target peptide sequences corresponding to one or more peptides expected to be present in the sample;
estimating, using one or more machine learning models, an elution order of the one or more expected peptides from a chromatography column;
initiating generation of a first set of mass spectrometry spectra for the sample;
during generation of the first set of mass spectrometry spectra, detecting peaks within the first set of mass spectrometry spectra to determine a real-time status with respect to the estimated elution order;
based on the determined real-time status with respect to the estimated elution order, selecting one or more peptide ions that are (i) observed in the first set of mass spectrometry spectra and (ii) included among the one or more peptides expected to be present in the sample; and
initiating generation of a second set of mass spectrometry spectra for the one or more selected peptide ions.

2. The method of claim 1, wherein identifying the one or more target peptide sequences for the sample comprises:
predicting, using one or more additional machine learning models, fragment intensities of mass spectrometry spectra of a plurality of peptides;
ranking the plurality of peptides based on a metric indicative of a variance of the predicted fragment intensities for each of the plurality peptides; and
selecting a subset of the plurality of peptides that has the lowest values of the metric.

3. The method of claim 1, comprising estimating a compensation voltage that maximizes sensitivity of a mass spectrometer to the peptide ions,
wherein selecting the one or more peptide ions that are (i) observed in the first set of mass spectrometry spectra and (ii) included among the one or more peptides expected to be present in the sample is additionally based on the compensation voltage.

4. (canceled)

5. (canceled)

6. The method of claim 1, wherein initiating generation of the first set of mass spectrometry spectra for the sample comprises generating a plurality of individual spectra having different mass-to-charge ranges, and wherein the different mass-to-charge ranges are optionally selected based on at least one of (i) the one or more target peptide sequences, (ii) the determined real-time status with respect to the estimated elution order, (iii) intensities of previously recorded signals in the given mass-to-charge ranges, or (iv) compensation voltage predictions.

7. (canceled)

8. The method of claim 1, wherein initiating generation of the second set of mass spectrometry spectra for the one or more selected peptide ions comprises defining a width of a mass-to-charge range for at least one spectrum of the second set of mass spectrometry spectra, the width being defined based on (i) intensities of signals in the first set of mass spectrometry spectra, (ii) a number of peptide ion signals in a given mass-to-charge range, and (iii) an estimated accumulation time required for collecting a threshold number of ions for each of the peptide ion signals in the given mass-to-charge range.

9. The method of claim 1, comprising analyzing the second set of mass spectrometry spectra, wherein the analyzing comprises inputting data indicative of the second set of mass spectrometry spectra into one or more convolutional neural networks trained to identify a presence of one or more peptides in the sample based on the data indicative of the second set of mass spectrometry spectra.

10. The method of claim 1, comprising selecting one or more fragment ions that are observed in the second set of mass spectrometry spectra; and initiating generation of a third set of mass spectrometry spectra for the one or more selected fragment ions, wherein the third set of mass spectrometry spectra is optionally generated by (i) isolating the one or more selected fragment ions, (ii) further fragmenting the one or more selected fragment ions to produce further fragmented ions, and (iii) detecting at least a portion of the further fragmented ions, wherein the further fragmented ions comprise isobaric tag reporter ions, and
wherein the method optionally comprises analyzing the third set of mass spectrometry spectra for the one or more selected fragment ions to quantify an amount of at least one detected peptide present in the sample.

11. (canceled)

12. The method of claim 10, wherein selecting the one or more fragment ions that are observed in the second set of mass spectrometry spectra comprises scoring the one or more fragment ions based on at least one of: (i) a correlation between predicted and observed fragment ion intensities, (ii) a deviation between predicted and observed retention times for the one or more expected peptides, (iii) a number of observed fragment ions relative to a number of fragment ions predicted to be observed, (iv) a mass accuracy of an observed peptide signal from the first set of mass spectrometry spectra, and (v) a score reflecting a match between observed and predicted data based on a background-normalized dot-product.

13. The method of claim 10, wherein initiating the generation of the third set of mass spectrometry spectra for the one or more selected fragment ions comprises:
estimating a time required for collecting a threshold amount of each of the one or more selected fragment ions that correspond to a single peptide, the threshold amount corresponding to a signal-to-noise threshold for isobaric tag reporter ion signals; and
initiating the generation of the third set of mass spectrometry spectra to collect data for at least the estimated time.

14. (canceled)

15. (canceled)

16. The method of claim 1, wherein initiating generation of the second set of mass spectrometry spectra for the one or more selected peptide ions comprises:
isolating the one or more selected peptide ions in a mass spectrometer that produces the mass spectrometry spectra,
fragmenting the one or more selected peptide ions to generate fragment ions, and
recording measurements related to at least a portion of the generated fragment ions.

17. A system comprising:
one or more computers and one or more storage devices on which are stored instructions that are operable, when executed by the one or more computers, to cause the one or more computers to perform operations comprising:
identifying one or more target peptide sequences for a sample, the one or more target peptide sequences corresponding to one or more peptides expected to be present in the sample;
estimating, using one or more machine learning models, an elution order of the one or more expected peptides from a chromatography column;
initiating generation of a first set of mass spectrometry spectra for the sample;
during generation of the first set of mass spectrometry spectra, detecting peaks within the first set of mass spectrometry spectra to determine a real-time status with respect to the estimated elution order;
based on the determined real-time status with respect to the estimated elution order, selecting one or more peptide ions that are (i) observed in the first set of mass spectrometry spectra and (ii) included among the one or more peptides expected to be present in the sample; and
initiating generation of a second set of mass spectrometry spectra for the one or more selected peptide ions.

18. The system of claim 17, wherein identifying the one or more target peptide sequences for the sample comprises:
predicting, using one or more additional machine learning models, fragment intensities of mass spectrometry spectra of a plurality of peptides;
ranking the plurality of peptides based on a metric indicative of a variance of the predicted fragment intensities for each of the plurality peptides; and
selecting a subset of the plurality of peptides that has the lowest values of the metric.

19. The system of claim 17, wherein the operations comprise estimating a compensation voltage that maximizes sensitivity of a mass spectrometer to the peptide ions,
wherein selecting the one or more peptide ions that are (i) observed in the first set of mass spectrometry spectra and (ii) included among the one or more peptides expected to be present in the sample is additionally based on the compensation voltage.

20. (canceled)

21. (canceled)

22. The system of claim 17, wherein initiating generation of the first set of mass spectrometry spectra for the sample comprises generating a plurality of individual spectra having different mass-to-charge ranges, and wherein the different mass-to-charge ranges are optionally selected based on at least one of (i) the one or more target peptide sequences, (ii) the determined real-time status with respect to the estimated elution order, (iii) intensities of previously recorded signals in the given mass-to-charge ranges, or (iv) compensation voltage predictions.

23. (canceled)

24. The system of claim 17, wherein initiating generation of the second set of mass spectrometry spectra for the one or more selected peptide ions comprises defining a width of a mass-to-charge range for at least one spectrum of the second set of mass spectrometry spectra, the width being defined based on (i) intensities of signals in the first set of mass spectrometry spectra, (ii) a number of peptide ion signals in a given mass-to-charge range, and (iii) an estimated accumulation time required for collecting a threshold number of ions for each of the peptide ion signals in the given mass-to-charge range.

25. The system of claim 17, wherein the operations comprise analyzing the second set of mass spectrometry spectra, wherein the analyzing comprises inputting data indicative of the second set of mass spectrometry spectra into one or more convolutional neural networks trained to identify a presence of one or more peptides in the sample based on the data indicative of the second set of mass spectrometry spectra.

26. The system of claim 17, wherein the operations comprise selecting one or more fragment ions that are observed in the second set of mass spectrometry spectra; and initiating generation of a third set of mass spectrometry spectra for the one or more selected fragment ions, wherein the third set of mass spectrometry spectra is optionally generated by (i) isolating the one or more selected fragment ions, (ii) further fragmenting the one or more selected fragment ions to produce further fragmented ions, and (iii) detecting at least a portion of the further fragmented ions, wherein the further fragmented ions comprise isobaric tag reporter ions, and
wherein the method optionally comprises analyzing the third set of mass spectrometry spectra for the one or more selected fragment ions to quantify an amount of at least one detected peptide present in the sample.

27. (canceled)

28. (canceled)

29. The system of claim 26, wherein initiating the generation of the third set of mass spectrometry spectra for the one or more selected fragment ions comprises:
estimating a time required for collecting a threshold amount of each of the one or more selected fragment ions that correspond to a single peptide, the threshold amount corresponding to a signal-to-noise threshold for isobaric tag reporter ion signals; and
initiating the generation of the third set of mass spectrometry spectra to collect data for at least the estimated time.

30. (canceled)

31. (canceled)

32. The system of claim 17, wherein initiating generation of the second set of mass spectrometry spectra for the one or more selected peptide ions comprises:
isolating the one or more selected peptide ions in a mass spectrometer that produces the mass spectrometry spectra,
fragmenting the one or more selected peptide ions to generate fragment ions, and
recording measurements related to at least a portion of the generated fragment ions.

33. (canceled)

34. One or more machine-readable storage devices having encoded thereon computer readable instructions for causing one or more processing devices to perform the method of claim 1.
